(19) United States
(12) Patent Application Publication
(10) Pub. No.: US 2011/0142945 A1
(43) Pub. Date: Jun. 16, 2011

(54) Hydrophobic Active Agent Compositions and Related Methods

(75) Inventors: Feng-Jing Chen, Salt Lake City, UT (US); Kathryn Gutke, Salt Lake City, UT (US); Srinivasan Venkateshwaran, Salt Lake City, UT (US); Mahesh V. Patel, Salt Lake City, UT (US)

(73) Assignee: Lipocine Inc.

(21) Appl. No.: 13/029,989

(22) Filed: Feb. 17, 2011

Related U.S. Application Data
(63) Continuation of application No. 10/322,344, filed on Dec. 17, 2002.

(51) Int. Cl. A61K 9/14; A61K 31/5685

(52) U.S. Cl. 424/489; 514/178

(57) ABSTRACT

Compositions and methods for providing hydrophobic active agents in a bioavailable form are disclosed and described. In one aspect of the invention, pharmaceutical composition containing a testosterone ester is provided. The composition includes a testosterone ester in both dissolved form and as undissolved particles and the dissolved form comprises at least 35 wt % of the testosterone ester present in the composition. The composition further includes a solubilizer and a stabilizer.

### PRIORITY DATA

This application is a continuation application of U.S. patent application Ser. No. 10/322,344, filed Dec. 17, 2002, which is incorporated herein by reference.

### FIELD OF THE INVENTION

The present invention relates to compositions containing a hydrophobic active agent, and methods associated therewith. Accordingly, the present invention involves the fields of chemistry, pharmaceutical sciences, and medicine.

### BACKGROUND

A number of hydrophobic drugs have been discovered to have a desirable, or potentially desirable effect in treating a variety of known conditions and maladies. For example, cyclosporine has been found to be very useful as an immune system suppressing agent, and is therefore indicated for administration to transplant patients in order to prevent the body from rejecting the newly received organ. However, the hydrophobic nature of such drugs causes significant challenges with administration and efficacy.

Specifically, because the contents of the human digestive tract are aqueous, drugs that are highly hydrophobic generally cannot become sufficiently solubilized to allow significant absorption through the digestive tract. As a result, most hydrophobic drugs suffer from poor oral bioavailability.

A wide variety of attempts have been made to improve the aqueous solubility of highly hydrophobic drugs. Modification of the drug itself into a more hydrophilic form while retaining the desired therapeutic effect, and delivery of the hydrophobic drug using a special carrier are probably the two most common approaches. Examples of specific carrier formulations used to improve aqueous solubility of various hydrophobic drugs may be found in U.S. Pat. Nos. 6,294,192, and 6,383,471, each of which are incorporated herein by reference. Additionally, a number of carriers have been formulated to specifically increase the aqueous solubility of cyclosporine, or to otherwise increase its in vivo bioavailability. Specific examples of such formulations may be found in U.S. Pat. Nos. 5,766,629, 5,798,333, and 6,008,192, as well as in PCT Publication Nos. WO 94/25068 and WO 98/33512, each of which is incorporated herein by reference.

Many attempts to increase the bioavailability of cyclosporine have focused on formulating cyclosporine in pre-concentrates wherein upon contact with an aqueous medium, cyclosporine remains completely solubilized in the resulting emulsion or microemulsion oil droplets. One major disadvantage of such formulations is the requirement of a significant amount of oil or other lipophilic material that is required in order to solubilize the cyclosporine and form almost entirely an emulsion or microemulsion upon contact with aqueous environment. Not only do such material pose an increased cost burden, but they also complicate manufacturing, and may raise issues that reduce freedom in formulating various dosages, such as stability issues, and interaction with other dosage ingredients.

As a result, cost effective cyclosporine compositions that are simple and present cyclosporine in a bioavailable form continue to be sought through ongoing research and development efforts.

### SUMMARY OF THE INVENTION

Accordingly, the present invention provides pharmaceutical compositions for poorly water soluble active agents that generally include a therapeutically effective amount of the active agent, a solubilizer, and a stabilizer in an amount sufficient to produce therapeutically relevant blood levels upon oral administration.

In one aspect, the present invention provides a pharmaceutical cyclosporine composition that includes a therapeutically effective amount of cyclosporine, a solubilizer of ethanol, and a stabilizer of a polyethoxylated castor oil, or a combination of a polyethoxylated castor oil and a polyethoxylated hydrogenated castor oil, in an amount sufficient to provide a ratio of stabilizer to cyclosporine of at least about 5:1. Such a composition has been found to form, upon contact with an aqueous medium, a bioavailable dispersion of dissolved cyclosporine and particles containing undissolved cyclosporine, with at least about 35% w/w of the cyclosporine being dissolved.

Additionally, the present invention encompasses an aqueous dispersion that provides cyclosporine in a substantially bioavailable form. In one aspect, such a dispersion may include a mixture of an ethanol solubilizer and a stabilizer of at least one polyethoxylated surfactant in an aqueous solution, and a therapeutically effective amount of cyclosporine contained in the dispersion. In one aspect, the cyclosporine is contained in the dispersion as both dissolved cyclosporine and particles of undissolved cyclosporine with at least about 35% w/w of the cyclosporine being dissolved, and wherein the amount of stabilizer is sufficient to provide a weight percentage or parts by weight ratio of stabilizer to cyclosporine of at least about 5:1.

The present invention additionally includes various methods associated with the above-recited compositions and dispersions. In one aspect, the present invention includes a method of treating a condition in a subject for which cyclosporine is indicated including the steps of: 1) providing a pharmaceutical cyclosporine composition as recited herein, and administering the composition to the subject in a therapeutically effective amount.

There has thus been outlined, rather broadly, various features of the present invention so that the detailed description thereof that follows may be better understood, and so that the present contribution to the art may be better appreciated. Other features of the present invention will become clearer from the following detailed description of the invention, taken with the accompanying claims, or may be learned by the practice of the invention.

### DETAILED DESCRIPTION OF THE INVENTION

Before the present compositions and methods are disclosed and described, it is to be understood that the present invention is not limited to the particular process steps and materials disclosed herein, but is extended to equivalents thereof as would be recognized by those ordinarily skilled in the relevant arts. It should also be understood that terminology employed herein is used for the purpose of describing particular embodiments only and is not intended to be limiting.

### DEFINITIONS

In describing and claiming the present invention, the following terminology will be used.

The singular forms “a,” “an,” and, “the” include plural referents unless the context clearly dictates otherwise. Thus, for example, reference to “the carrier” includes reference to one or more specific carriers, reference to “an additive” includes reference to one or more of such additives, and reference to “the plasticizing agent” includes reference to one or more of such agents.

The terms “composition” and “formulation may be used interchangeably herein.

The terms “lipophilic” and “hydrophobic” may be used interchangeably herein. Moreover, when used in connection with a drug, such terms refer to a drug having an aqueous solubility of less than about 1 mg/ml at 25° C. in a completely solubilized form.

As used herein, an “effective amount,” and “sufficient amount” may be used interchangeably, and refer to an amount of a substance that is sufficient to achieve an intended purpose or objective. For example, a sufficient, or effective amount of a suspending agent would be the minimum amount of agent required to effectively suspend one substance, such as a pharmaceutically active agent, in a carrier. As a result, the term “therapeutically effective amount” refers to an amount of a pharmaceutically active agent that is sufficient to achieve a desired therapeutic effect or result, when administered to a subject. The determination of effective and therapeutically effective amounts is well within the knowledge of one ordinarily skilled in the pharmaceutical, and medical sciences. See, for example, Meiner and Tonascia, “Clinical Trials: Design, Conduct, and Analysis,” Monographs in Epidemiology and Biostatistics, Vol. 8 (1986).

As used herein, “pharmaceutically active agent,” “bioactive agent,” “therapeutic agent,” “active agent,” and “drug” may be used interchangeably, and refer to a substance, such as a chemical compound or complex, that has a measurable beneficial physiological effect on the body, such as a therapeutic effect in treatment of a disease or disorder, when administered in an effective amount. Further, when these terms are used, or when a particular active agent is specifically identified by name or category, it is to be understood that such recitation is intended to include the active agent per se, as well as pharmaceutically acceptable, pharmacologically active derivatives thereof, or compounds significantly related thereto, including without limitation, salts, esters, amides, prodrugs, active metabolites, isomers, fragments, analogs, etc.

As used herein, “bioavailable” refers to the ability of a compound, such as a therapeutic agent to pass through a biological membrane for absorption into the body. As is known in the art, the bioavailability of a specific compound is generally dictated by its chemical and physical properties, such as its relative lipophilicity. Moreover, various mechanisms of determining and quantifying the bioavailability of a given compound are also known to those of ordinary skill in the art, such as by evaluating the area under the curve (AUC) and Cₘₐₓ values of the specific compound in the blood serum of a subject to whom the compound has been administered. Additional information relevant to the determination of bioavailability, may be found on pages 605-612 of Remington: The Science and Practice of Pharmacy 19^{th} ed. (1995). Further, specific information about the bioavailability of various currently approved cyclosporine products, such as those sold by Novartis Pharmaceuticals Corporation (East Hanover, N.J.) under the trade name Neoral® may be found on pages 2380-2387 of The Physicians' Desk Reference, 56^{th} ed. (2002), which is incorporated herein by reference.

As used herein, “cyclosporin” refers to a group of nonpolar cyclic oligo-peptides with immunosuppressant activity. Various specific cyclosporins, including Cyclosporin A, Cyclosporin B, Cyclosporin C, Cyclosporin D, and Cyclosporin G, are well known to those in the art. Cyclosporin is contained as entry 2821 on page 464 of The Merck Index 12^{th} ed. (1996), which is incorporated herein by reference. Further, it is to be understood that as used herein “cyclosporine” refers to Cyclosporin A, as is well known by those of ordinary skill in the art.

As used herein, “dissolved” and “solubilized” may be used interchangeably, and refer to a substance or compound that when mixed with a liquid, forms a solution or a dispersion with substantially no solid (crystalline or amorphous) fraction. In one aspect, such properties may include compartmentalization of a compound within a micelle or other structure that creates droplets containing the dissolved substance in a bulk aqueous phase.

As used herein “undissolved” refers to a substance or compound that is in a solid state that is of high degree of order. The structure of the solid may be crystalline and lattice-like or noncrystalline (amorphous), such as plastic, glass or gels which are not lattice-like or only partly so in its dispersing medium, typically an aqueous medium. For example, see page 184 of Remington: The Science and Practice of Pharmacy 17^{th} ed., 1985. As is understood by those of ordinary skill in the art, this undissolved state as such is typically unamenable to the absorption of the particles across a biological membrane. In one aspect, an undissolved substance or compound may be in particulate form and include a plurality of particles.

As used herein, “solubilizer” refers to an agent that is capable of fully or partially dissolving or solubilizing a drug or pharmaceutically active agent.

As used herein, “stabilizer” refers to a lipidic agent that is capable of forming an aqueous dispersion of undissolved particles of a hydrophobic active agent and preventing the aggregation and/or settling of such particles for a time period that is relevant to the absorption of the active agent. In one aspect, the stabilizer may be a surfactant.

As used herein, “hydrophilic-lipophilic balance (HLB) value” refers to an empirical parameter used to characterize the relative hydrophilicity or lipophilicity of an amphiphilic compound, such as a surfactant. The determination of HLB is well within the ability of one ordinarily skilled in the art. However, it should be appreciated that the HLB value of a surfactant is merely a rough guide generally used to enable the formulation of various products. For many important surfactants, including several polyethoxylated surfactants, it has been reported that HLB values can differ by as much as about 8 HLB units, depending upon the empirical method chosen to determine the HLB value. See, Schott, J. Pharm. Sciences, 79(1), 87-88 (1990)), which is incorporated herein by reference.

As used herein, “hydrophilic surfactant” refers to a surfactant having an HLB value of greater than about 10.

As used herein, “lipophilic surfactant” refers to a surfactant having an HLB value of less than about 10. Notably, not all lipophilic compounds are surfactants.

Concentrations, amounts, solubilities, particle size, wavelength, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited.

As an illustration, a concentration range of “about 4% w/w to about 60% w/w” should be interpreted to include not only the explicitly recited concentration of about 4% w/w to about 60% w/w, but also include individual concentrations and the sub-ranges within the indicated range. Thus, included in this numerical range are individual concentrations such as 4% w/w, 10% w/w, 23% w/w, and 46% w/w, and sub-ranges such as from 10% w/w to 50% w/w, from 20% w/w to 40% w/w, from 25% w/w to 35% w/w, from 15% w/w to 20% w/w, etc.

This same principle applies to ranges reciting only one numerical value. Furthermore, such an interpretation should apply regardless of the breadth of the range or the characteristics being described.

### INVENTION

Applicants have discovered that various hydrophobic drugs may be rendered adequately bioavailable, even though they are not completely solubilized or dissolved in an aqueous medium. One mechanism for effecting such bioavailability is through the employment of an aqueous dispersion which comprises dissolved hydrophobic drug and particles containing undissolved hydrophobic drug in an aqueous medium or a composition or a pre-concentrate which is capable of forming such aqueous dispersion in situ. Generally, such a dispersion includes a number of components besides the aqueous medium, including a mixture of a solubilizer and a stabilizer. Further, such a dispersion contains a therapeutically effective amount of the hydrophobic drug in both dissolved form, and as undissolved particles, with at least about 35% w/w of the dispersion being dissolved drug. Additionally, the present invention extends to various compositions or pre-concentrates that are capable of forming such bioavailable dispersions, and to methods for the use thereof as set forth more fully below. Such compositions or pre-concentrates comprise a mixture of a solubilizer, a stabilizer, and a therapeutically effective amount of a hydrophobic drug at least partially dissolved in said composition or pre-concentrates. In another aspect, the hydrophobic drug may be completely solubilized in the mixture of the solubilizer and stabilizer of the pre-concentrates or the compositions.

A. Therapeutic Agents

It has been found that a variety of hydrophobic drugs can be made effectively bioavailable without being completely solubilized by using the dispersions of the present invention. Examples of such drugs include without limitation: 3-ketodesogestrel, 4-dihydrotestosterone, abecarnil, acamprostate, acavir, acebutolol, aceclofenac, acemetacin, acetaminophen, acetaminosalol, acetanilide, acetohexamide, acetophenazine maleate, acetophenazine, acetoxolone, acetoxypregnenolone, acetretin, acrisorcin, acrivastine, acyclovir, adinazolam, adiphenine hydrochloride, adrafinil, adrenolone, agatroban, ahnitrine, akatinol, alatrofloxacin, albendazole, albuterol, aldioxa, alendronate, alfentanil, alib endol, alitretinoin, allopurinol, allylamines, allylestrenol, alminoprofen, almotriptan, alosetron, aloxiprin, alprazolam, alprenolol, amantadine, ambucetamide, amidephrine, amidinomycin, amiloride, aminoarylcarboxylic acid derivatives, aminoglutethimide, aminoglycosides, aminopentamide, aminopromazine, a minorex, amiodarone, amiphenazole, amiprilose, amisulpride, amitriptyline, amlexanox, amlodipine, amodiaquine, amosulalol, amotriphene, amoxapine, amoxicillin, amphecloral, amphetamine, amphomycin, amphotericin, ampicillin, ampiroxicam, amprenavir, aminone, amsacrine, amyl nitrate, amylobarbitone, anagestone acetate, anastrozole, andinocillin, androstenediol, androstenediol-17-acetate, androstenediol-17-benzoate, androstenediol-3-acetate, androstenediol-3-acetate-17-benzoate, androstenedione, androsterone acetate, androsterone benzoate, androsterone propionate, androsterone, angiotensin, anidulafungin, aniracetam, apazone, apicycline, apoatropine, apomorphine, apraclonidine, aprepitant, aprotinin, arbaprostil, ardeparin, aripiprazole, arnikacin, arotinolol, arstiinol, arylacetic acid derivatives, arylalkylamines, arylbutyric acid derivatives, arylcarboxylic acids, arylpiperazines, arylpropionic acid derivatives, aspirin, astemizole, atenolol, atomoxetine, atorvastatin, atovaquone, atropine, auranofin, azapropazone, azathioprine, azelastine, azetazolamide, azithromycin, baclofen, bambuterol, bamethan, barbitone, barnidipine, basalazide, beclamide, beclobrate, beclomethasone, befimolol, bemegride, benazepril, bencyclane, bendazac, bendazol, bendroflumethiazide, benethamine penicillin, benexate hydrochloride, benfurodil hemisuccinate, benidipine, benorylate, bentazepam, benzhexyl, benziodarone, benznidazole, benzoctamine, benzodiazepine derivatives, benzodiazepine, benzonatate, benzphetamine, benzylmorphine, beperiden, bephenium hydroxynaphthoate, bepridil, bepridil, betahistine, betamethasone, betaxolol, bevantolol, bevonium methyl sulfate, bexarotene, bezafibrate, ialamicol, biapenem, bicalutamide, bietamiverine, bifonazole, binedaline, binifibrate, biricodar, bisacodyl, bisantrene, bisoprolol, bitolterol, bopindolol, boswellic acid, bradykinin, bretylium, bromazepam, bromocriptine, bromperidol, brotizolam, brovincamine, buciclate, bucloxic acid, bucumolol, budesonide, budralazine, bufeniode, bufetolol, buflomedil, bufuralol, bumetanide, bunitrolol, bupranolol, buprenorphine, buproprion, buspirone, busulfan, butalamine, butarphenol, butaverine, butenafine, butenafine, butidrine hydrochloride, butobarbitone, butoconazole nitrate, butoconazole, butofilol, butorphenol, butropium bromide, cabergoline, calcifediol, calcipotriene, calcitriol, caldiribine, cambendazole, camioxirole, camostat, camptothecin, candesartan, candoxatril, capecitabine, caprate, capsaicin, captopril, carazolol, carbacephems, carbamates, carbamezepine, carbapenems, carbarsone, carbatrol, carbenoxolone, carbimazole, carbromal, carbuterol, carisoprodol, carotenes, caroverine, carteolol, carvedilol, cefaclor, cefazolin, cefbuperazone, cefepime, cefoselis, ceftibuten, celcoxib, celecoxib, celiprolol, cephaeline, cephalosporin C, cephalosporins, cephamycins, cerivastatin, certoparin, cetamolol, cetiedil, cetirizine, cetraxate, chloracizine, chlorambucil, chlorbetamide, chlordantoin, chlordiazepoxide, chlormadinone acetate, chlormethiazole, chloroquine, chlorothiazide, chlorpheniramine, chlorphenoxamide, chlorphentermine, chlorproguanil, chlorpromazine, chlorpropamide, chlorprothixene, chlortetracycline, chlorthalidone, cholecalciferol, chromonar, ciclesonide, ciclonicate, cidofivir, ciglitazone, cilansetron, cilostazol, cimetidine, cimetropium bromide, cinepazet maleate, cinnamedrine, cinnarizine, cinolazepam, cinoxacin, ciprofibrate, ciprofloxacin, cisapride, cisplatin, citalopram, citicoline, clarithromycin, clebopride, clemastine, clenbuterol, clidanac, clinofibrate, clioquinol, clobazam, clobenfurol, clobenzorex, clofazimine, clofibrate, clofibric acid, cloforex, clomipramine, clonazepam, clonidine, clonitrate, clopidogrel, clopirac indomethacin, cloranolol, cloricromen, clorprenaline, clortermine, clotiazepam, clotrimazole, cloxacillin, clozapine, cmepazide, codeine methyl bromide, codeine phosphate, codeine sulfate, codeine, colloidal bismuth subcitrate, Complete List, cortisone, cromafiban, cromolyn, cropropamide, crotethamide, curcumin, cyclandelate, cyclarbamate, cyclazocine, cyclexedrine, cyclizine, cyclobenzaprine, cyclodrine, cyclonium iodide, cyclopentamine, cyclosporine, cypionate, cyproheptadine, cyproterone acetate, cyproterone, cytarabine, dacarbazine, dalfopristine, dantrolene sodium, dapiprazole, darodipine, decanoate, decitabine, decoquinate, dehydroemetine, dehydroepiandrosterone, delavirdine, delaviridine, demeclocycline, denopamine, deramciclone, descitalopram, desipramine, desloratadine, desogestrel, desomorphine, desoxymethasone, detomidine, dexamethasone, dexamphetamine, dexanabinol, dexchlorpheniramine, dexfenfluramine, dexmethylphenidate, dexrazoxane, dextroamphetamine sulfate, dextroamphetamine, dextropropoxyphene, DHEA, diacetate, diamorphine, diazemine, diazepam, diazoxide, dibromopropamidine, dichlorophen, diclofenac, dicoumarol, didanosine, dideoxyadenosine, diethylpropion, difemerine, difenamizole, diflunisal, digitoxin, digoxin, dihidroergotamine, dihydrocodeine, dihydrocodeinone enol acetate, dihydroergotamine mesylate, dihydroergotamine, dihydrogesterone, dihydromorphine, dihydropyridine derivatives, dihydrostreptomycin, dihydrotachysterol, dihydroxyaluminum acetylsalicylate, diiodohydroxyquinoline, diisopromine, dilazep, dilevalol, dilitazem, diloxanide furoate, diloxanide, diltiazem, dimefline, dimenhydrinate, dimethisterone, dimetofrine, dimorpholamine, dinitolmide, dioxaphetyl butyrate, dioxethedrine, diphemethoxidine, diphenhydramine, diphenoxylate, diphetarsone, dipivefrin, diponium bromide, dipyridamole, dirithromycin, disopyramide, divalproex sodium, dofetilide, domperidone, donezepil, dopexamine, dopradil, dosmalfate, doxapram, doxazosin, doxefazepam, doxepin, doxycycline, drofenine, dromostanolone propionate, dromostanolone, dronabinol, droperidol, droprenilamine, d-threo-methylphenidate, duloxetine, dutasteride, ebrotidine, eburnamonine, ecabet, ecenofloxacin, econazole nitrate, edavarone, edoxudine, efavirenz, effivarenz, efloxate, eledoisin, eletriptan, elgodipine, ellipticine, emepronium bromide, emetine, enalapril, enanthate, encamide, enlopitat, enoximone, enprostil, entacapone, epanolol, ephedrine, epinastine, epinephrine, epirubicin, epleronone, eposartan, ergocalciferol, ergoloid mesylates, ergotamine, ertapenum, erythromycin, erytlirityl tetranitrate, esaprazole, escitalopram, esmolol, esomeprazole, esonarimod, estazolam, estradiol benzoate, estradiol, estramustine, estriol succinate, estriol, estrone acetate, estrone sulfate, etafedrine, etafenone, ethacrynic acid, ethamivan, ethinamate, ethinylestradiol 3-acetate, ethinylestradiol 3-benzoate, ethinylestradiol, ethionamide, ethisterone (17α-ethinyltestosterone), ethopropazine, ethotoin, ethoxyphenamine, ethylestrenol, ethylmorphine, ethylnorepinephrine, ethynodiol diacetate, etodolac, etofibrate, etoposide, etoricoxib, etretinate, everolimus, exalamide, examestane, examorelin, ezemitibe, falecalcitriol, famciclovir, famotidine, fantofarone, farapenum, farglitazar, fasudil, felbamate, felodipine, fenalamide, fenbufen, fenbutrazate, fendiline, fenfluramine, fenofibrate, fenofibric acid, fenoldopam, fenoprofen, fenoterol, fenoverine, fenoxazoline, fenoxedil, fenpiprane, fenproporex, fenspiride, fentanyl, fexofenadine, flavoxate, flecamide, flopropione, floredil, floxuridine, fluconazole, flucytosine, fludarabine, fludiazepam, fludrocortisone, flufenamic acid, flunanisone, flunarizine, flunisolide, flunitrazepam, fluocortolone, fluoxetine, flupenthixol decanoate, fluphenazine decanoate, fluphenazine enanthate, fluphenazine, fluproquazone, flurazepam, flurbiprofen, fluorogestone acetate, fluticasone propionate, fluvastatin, fluvoxamine, fominoben, formoterol, foscarnet, foscarnet, fosinopril, fosphenyloin, frovatirptan, fudosteine, fumagillin, furazolidone, furazolidone, furfurylmethyl amphetamine, furosemide, gabapentin, gabexate, gaboxadol, galanthamine, gallopamil, gammaparin, ganciclovir, ganglefene, gefarnate, gemcitabine, gemfibrozil, gepirone, gestadene, ghrelin, glatiramer, glaucarubin, glibenclamide, gliclazide, glimepiride, glipizide, gluconic acid, glutamicacid, glyburide, glyceryl trinitrate, glymepiride, granisetron, grepafloxacin, griseofulvin, guaiazulene, guanabenz, guanfacine, halofantrine, haloperidol decanoate, haloperidol, haloxazolam, hepronicate, heptanoate, hexobendine, hexoprenaline, hydramitrazine, hydrazides, hydrochlorothiazide, hydrocodone, hydrocortisone, hydromorphone, hydroxyamphetamine, hydroxymethylprogesterone acetate, hydroxymethylprogesterone, hydroxyprogesterone acetate, hydroxyprogesterone caproate, hydroxyprogesterone, hymecromone, hyoscyamine, ibopamine, ibudilast, ibufenac, ibuprofen, ibutilide, idebenone, idoxuridine, ifenprodil, igmesine, iloprost, imatinib, imidapril, imidazoles, imipenem, imipramine, imolamine, incadronic acid pergolide, indanazoline, indenolol, indinavir, indomethacin, indoramin, inosinepranobex, inositol niacinate, iodoquinol, ipidracine, iproniazid, irbesartan, irinotecan, irsogladine, isobutyrate, isocaprate esters, isoetharine, isometheptene, isoproterenol, isosorbide dinitrate, isosorbide mononitrate, isosorbide dinitrate, isoxsuprine, isradipine, itasetron, itraconazole, itramintosylate, ivermectin, kallidin, kallikrein, kanamycin, ketamine, ketoconazole, ketoprofen, ketorolac, ketotifen, labetalol, lafutidine, lamifiban, lamivudine, lamotrigine, lanatoside c, lansoprazole, lasofoxifene, leflunomide, leminoprazole, lercanadipine, lesopitron, letrozole, leucovorin, levalbuterol, levallorphan, levetiracetam, levetriacetam, levobunolol, levodopa, levofloxacin, levonorgestrel, levophacetoperane, levorphanol, lidocaine, lidoflazine, lifibrol, limaprost, linezolid, lintitript, liranaftate, lisinopril, lisuride, lobeline, lobucavir, lodoxamide, lomefloxacin, lomerizine, lomustine, loperamide, lopinavir, loprazolam, loracarbef, loratadine, lorazepam, lorefloxacin, lormetazepam, losartan, lovasatain, lovastatin, loxapine succinate, loxapine, 1-threo-methylphenidate, lumiracoxib, lynestrenol, lysine acetylsalicylate, lysozyme, lysuride, mabuterol, mafenide, magnesium acetylsalicylate, malgramostin, mannitol hexanitrate, maprotiline, mazindol, mebendazole, meclizine, meclofenamic acid, mecloxaminepentapiperide, medazepam, medibazine, medigoxin, medrogestone, medroxyprogesterone acetate, mefenamic acid, mefenorex, mefloquin, mefloquine, megestrol acetate, megestrol, melengestrol acetate, melphalan, mematine, mepenzolate bromide, meperidine, mephenoxalone, mephentermine, mepindolol, mepixanox, meprobamate, meptazinol, mercaptopurine, merropenum, mesalamine, mesalazine, mesoridazine besylate, mesoridazine, mestranol, metaclazepam, metamfepramone, metampicillin, metaproterenol, metaraminol, methacycline, methadone hydrochloride, methadone, methamphetamine, methaqualone, metharnphetamine, methoin, methotrexate, methoxamine, methsuximide, methylhexaneamine, methylphenidate d-threo-methylphenidate, methylphenidate, methylphenobarbitone, methylprednisolone, methysergide, metiazinic acid, metizoline, metoclopramide, metolazone, metoprolol, metoxalone, metripranolol, metronidazole, mexiletine, mexilitene, mianserin, mibefradil, miconazole, midazolam, midodrine, miglitol, milnacipran, milrinone, minoxidil, mirtazapine, misoprostol, mitomycin, mitotane, mitoxantrone, mizolastine, modafinil, mofebutazone, mofetil, molindone hydrochloride, molindone, molsidomine, monatepil, montelukast, monteplase, moprolol, moricizine, morphine hydrochloride, morphine sulfate, morphine, morpholine salicylate, mosapramine, moxifloxacin, moxisylvyte, moxonidine, mycophenolate, nabumetone, nadolol, nadoxolol, nadroparin, nafamostat, nafronyl, naftopidil, nalbuphine, nalidixic acid, nalmefene, nalorphine, naloxone, naltrexone, nandrolone benzoate, nandrolone cyclohexanecarboxylate, nandrolone cyclohexane-propionate, nandrolone decanoate, nandrolone furylpropionate, nandrolone phenpropionate, naphazoline, naproxen, naratriptan, natamycin, nateglinide, nebivalol, nedocromil, nefazodone, nefopam, nelfinavir, nemonapride, neomycin undecylenate, neomycin, neotrofin, nesiritide, n-ethylamphetamine, nevibulol, nevirapine, nexopamil, nicametate, nicardipine, nicergoline, nicofibrate, nicofuranose, nicomorphine, nicorandil, nicotinyl alcohol, nicoumalone, nifedipine, nifenalol, nikethamide, nilutamide, nilvadipine, nimodipine, nimorazole, nipradilol, nisoldipine, nitisonone, nitrazepam, nitrofurantoin, nitrofurazone, nitroglycerin, nizatidine, norastemizole, norepinephrine, norethindrone acetate, norethindrone, norethisterone acetate, norethisterone, norethynodrel, norfenefrine, norfloxacin, norgestimate, norgestrel, norgestrienone, normethadone, normethisterone, normorphine, norpseudoephedrine, nortriptyline, novantrone, nylidrin, nystatin, octamylamine, octodrine, octopamine, ofloxacin, olanzapine, olanzapine, olapatadine, olmesartan, olopatidine, olsalazine, omapatrilat, omeprazole, ondasetron, opium, oprevelkin, orlistat, ornidazole, ornoprostil, oseltamivir, oxaliplatin, oxamniquine, oxandrolone, oxantel embonate, oxaprozin, oxatomide pemirolast, oxatomide, oxazepam, oxcarbazepine, oxfendazole, oxiconazole, oxiracetam, oxolinicacid, oxprenolol, oxycodone, oxyfedrine, oxymetazoline, oxymorphone, oxyphenbutazone, oxyphencyclimine, oxyprenolol, ozagrel, paclitaxel, palonosetron, pantoprazole, papaverine, paracalcitol, paramethadione, parecoxib, pariprazole, paromomycin, paroxetine, parsalmide, pazinaclone, pemoline, penbutolol, penciclovir, penicillin G benzathine, penicillin G procaine, penicillin V, penicillins, pentaerythritol tetranitrate, pentaerythritol tetranitrate, pentapiperide, pentazocine, pentifylline, pentigetide, pentobarbitone, pentorex, pentoxifylline, pentrinitrol, perbuterol, perenzepine, pergolide, perhexyline, perindopril erbumine, perospirone, perphenazine pimozide, perphenazine, phanquinone, phenacemide, phenacetin, phenazopyridine, phencarbamide, phendimetrazine, phenelzine, phenindione, phenmetrazine, phenobarbitone, phenoperidine, phenothiazines, phenoxybenzamine, phensuximide, phentermine, phentolamine, phenyl salicylate, phenylacetate, phenylbutazone, phenylephrinehydrochloride, phenylpropanolamine hydrochloride, phenylpropanolaminehydrochloride, phenylpropyl-methylamine, phenyloin, phloroglucinol, pholedrine, physostigmine salicylate, physostigmine, phytonadiol, piapenum, picilorex, piclamilast, picrotoxin, picumast, pifamine, pilsicamide, pimagedine, pimeclone, pimecrolimus, pimethylline, pimozide, pinaverium bromide, pindolol, pioglitazone, piperacillin, piperazine estrone sulfate, piperazine derivatives, piperilate, piracetam, pirbuterol, pirenzepine, piribedil, pirifibrate, piroxicam, pitavastatin, pizotyline, plaunotol, polaprezinc, polybenzarsol, polyestrol phosphate, practolol, pralnacasan, pramipexole, pranlukast, prasterone, pravastatin, prazepam, praziquantel, prazosin, prednisolone, prednisone, pregabalin, prenalterol, prenylamine, pridinol, prifinium bromide, primidone, primipramine, probenecid, probucol, procainamide, procarbazine, procaterol, prochlorperazine, progesterone, proguanil, pronethalol, propafenone, propamidine, propatyl nitrate, propentoffyline, propionate, propiram, propoxyphene, propranolol, propylhexedrine, propylthiouracil, protokylol, protriptyline, proxazole, pseudoephedrine, purines, pyrantel embonate, pyrazoles, pyrazolones, pyridofylline, pyrimethamine, pyrimidines, pyrrolidones, quazepam, quetiapine, quetuapine, quinagolide, quinapril, quinestrol, quinfamide, quinidine, quinine sulfate, quinolones, quinupritin, rabalzotan, rabeprazole sodium, rabeprazole, racefimine, ramatroban, ramipril, ranitidine, ranolazine, ransoprazole, rasagiline, rebamipide, refludan, repaglinide, repinotan, repirinast, reproterol, reserpine, retinoids, ribavirin, rifabutine, rifampicin, rifapentine, rilmenidine, riluzole, rimantadine, rimiterol, rioprostil, risperidone, ritanovir, ritapentine, ritipenem, ritodrine, ritonavir, rivastigmine, rizatriptan, rociverine, rofecoxib, rohypnol, rolipram, romoxipride, ronifibrate, ropinirole, ropivacaine, rosaprostol, rosiglitazone, rosuvastatin, rotinolol, rotraxate, roxatidine acetate, roxindole, rubitecan, salacetamide, salicin, salicylamide, salicylic acid derivatives, salmeterol, saquinavir, saquinavir, scopolamine, secnidazole, selegiline, semotiadil, seratrodast, sertindole, sertraline, sibutramine, sildenafil, simfibrate, simvastatin, siramesine, sirolimus, sitaxsentan, sofalcone, somotiadil, sorivudine, sotalol, soterenol, sparfloxacin, spasmolytol, spectinomycin, spiramycin, spironolactone, spizofurone, stanozolol, stavudine, streptomycin, succinylsulfathiazole, sucralfate, sufentanil, sulconazole nitrate, sulfacetamide, sulfadiazine, sulfaloxicacid, sulfarside, sulfinalol, sulindac, suloctidil, sulphabenzamide, sulphacetamide, sulphadiazine, sulphadoxine, sulphafurazole, sulphamerazine, sulphamethoxazole, sulphapyridine, sulphasalazine, sulphinpyrazone, sulpiride, sulthiame, sultopride, sultroponium, sumanirole, sumatriptan, sunepitron, superoxide dismutase, suplatast, suramin sodium, synephrine, tacrine, tacrolimus, tacrolimus, tadalafil, talinolol, talipexole, tamoxifen, tamsulosin, targretin, tazanolast, tazarotene, tazobactum, tecastimezole, teclozan, tedisamil, tegaserod, telenzepine, telmisartan, temazepam, teniposide, teprenone, terazosin, terbenafine, terbinafine, terbutaline sulfate, terbutaline, terconazole, terfenadine, terodiline, terofenamate, tertatolol, testolactone, testosterone, tetracyclics, tetracycline, tetrahydrocannabinol, tetrahydrozoline, thalidomide, theofibrate, thiabendazole, thiazinecarboxamides, thiocarbamates, thiocarbamizine, thiocarbarsone, thioridazine, thiothixene, tiagabine, tiamenidine, tianeptine, tiaprofenic acid, tiaramide, ticlopidine, tigloidine, tilisolol, timolol, timidazole, tinofedrine, tinzaparin, tioconazole, tipranavir, tirapazamine, tirofiban, tiropramide, titanicene, tizanadine, tizanidine, tizinadine, tocamide, tolazamide, tolazoline, tolbutamide, tolcapone, tolciclate, tolfenamic acid, toliprolol, tolteridine, tolterodine, tonaberstat, topiramate, topotecan, torasemide, toremifene citrate, toremifene, tosufloxacin, tramadol, tramazoline, trandolapril, tranilast, tranylcypromine, trapidil, traxanox, trazodone, tretoquinol, triacetin, triamcinolone, triampterine, triamterine, triazolam, triazoles, tricromyl, tricyclics, trifluoperazine hydrochloride, trifluoperazine, triflupromazine, trifluridine, trihexyphenidyl hydrochloride, trihexyphenidyl, trimazosin, trimebutine, trimetazidine, trimethoprim, trimgestone, trimipramine, trimoprostil, trithiozine, troglitazone, troInitrate phosphate, tromethamine, tropicamide, trovafloxacin, troxipide, tuaminoheptane, tulobuterol, tymazoline, tyramine, undecanoate, undecanoic acid, urinastatin, ursodeoxycholic acid, valacyclovir, valdecoxib, valerate, valganciclovir, valproic acid, valsartan, vancomycin, vardenafil, venlafaxine, venorelbine, verapamil, verapimil, vidarabine, vigabatrin, vincamine, vinpocetine, viomycin, viquidil, visnadine, vitamin a derivatives, vitamin a, vitamin b2, vitamin d, vitamine, vitamin k, voglibose, voriconazole, xaliproden, xamoterol, xanthinol niacinate, xenytropium bromide, xibenolol, ximelagatran, xylometazoline, yohimbine, zacopride, zafirlukast, zafirlukat, zalcitabine, zaleplon, zanamivir, zatebradine, ziconotide, zidovudine, zileuton, zimeldine, zinc propionate, ziprasidone, zolimidine, zolmitriptan, zolpidem, zonisamide, zopiclone. The more preferred active agents include alendronate, amiodarone, amlodipine, amprenavir, anastrozole, aprepitant, aripiprazole, atomoxetine, atorvastatin, atovaquone, azathioprine, azelastine, azithromycin, bicalutamide, budesonide, buproprion, butarphenol, butorphenol, candesartan, carbamezepine, carisoprodol, carvedilol, celcoxib, cetirizine, ciclesonide, cilostazol, clopidogrel, cyclobenzaprine, delaviridine, deramciclone, descitalopram, desloratadine, DHEA, didanosine, dihidroergotamine, dipyridamole, donezepil, dronabinol, duloxetine, dutasteride, effivarenz, enlopitat, entacapone, epirubicin, ergotamine, etoricoxib, everolimus, ezemitibe, felodipine, fentanyl, frovatirptan, gabapentin, granisetron, halofantrine, hydrocodone, itasetron, lamotrigine, lansoprazole, leflunomide, lercanadipine, letrozole, letrozole, levetiracetam, lovasatain, lumiracoxib, malgramostin, mefloquin, mematine, mesalamine, metolazone, mirtazapine, modafinil, nefazodone, nelfinavir, nifedipine, nilutamide, nimodipine, nisoldipine, norastemizole, norfloxacin, olanzapine, olapatadine, ondasetron, oxaprozin, oxcarbazepine, oxycodone, perbuterol, phenazopyridine, pimecrolimus, pioglitazone, pralnacasan, prasterone, pravastatin, propafenone, quetuapine, repaglinide, riluzole, risperidone, ritanovir, rivastigmine, rofecoxib, saquinavir, sertindole, sertraline, sildenafil, simvastatin, sirolimus, spironolactone, stavudine, sumatriptan, tacrolimus, tadalafil, tamsulosin, tazarotene, tazarotene, tecastimezole, tegaserod, terbenafine, thalidomide, tiagabine, tizanadine, tizinadine, tolcapone, tolteridine, topiramate, torasemide, toremifene, tramadol, valdecoxib, valproic acid, vardenafil, vigabatrin, voriconazole, ximelagatran, zafirlukat, zaleplon, zileuton, ziprasidone, zolpidem, zonisamide.

In a more detailed aspect, the hydrophobic drug may be a member selected from the group consisting essentially of: alendronate, amiodarone, amlodipine, amprenavir, anastrozole, aprepitant, aripiprazole, atomoxetine, atorvastatin, atovaquone, azathioprine, azelastine, azithromycin, bicalutamide, budesonide, buproprion, butarphenol, butorphenol, candesartan, carbamezepine, carisoprodol, carvedilol, celcoxib, cetirizine, ciclesonide, cilostazol, clopidogrel, cyclobenzaprine, delaviridine, deramciclone, descitalopram, desloratadine, DHEA, didanosine, dihidroergotamine, dipyridamole, donezepil, dronabinol, duloxetine, dutasteride, effivarenz, enlopitat, entacapone, epirubicin, ergotamine, etoricoxib, everolimus, ezemitibe, felodipine, fentanyl, frovatirptan, gabapentin, granisetron, halofantrine, hydrocodone, itasetron, lamotrigine, lansoprazole, leflunomide, lercanadipine, letrozole, letrozole, levetiracetam, lovasatain, lumiracoxib, malgramostin, mefloquin, mematine, mesalamine, metolazone, mirtazapine, modafinil, nefazodone, nelfinavir, nifedipine, nilutamide, nimodipine, nisoldipine, norastemizole, norfloxacin, olanzapine, olapatadine, ondasetron, oxaprozin, oxcarbazepine, oxycodone, perbuterol, phenazopyridine, pimecrolimus, pioglitazone, pralnacasan, prasterone, pravastatin, propafenone, quetuapine, repaglinide, riluzole, risperidone, ritanovir, rivastigmine, rofecoxib, saquinavir, sertindole, sertraline, sildenafil, simvastatin, sirolimus, spironolactone, stavudine, sumatriptan, tacrolimus, tadalafil, tamsulosin, tazarotene, tazarotene, tecastimezole, tegaserod, terbenafine, thalidomide, tiagabine, tizanadine, tizinadine, tolcapone, tolteridine, topiramate, torasemide, toremifene, tramadol, valdecoxib, valproic acid, vardenafil, vigabatrin, voriconazole, ximelagatran, zafirlukat, zaleplon, zileuton, ziprasidone, zolpidem, and zonisamide, and mixtures thereof. In another aspect, the drug may be cyclosporine.

The specific amount and type of drug selected for use in the dispersions of the present invention may be dictated by various considerations, such as other specific dispersion ingredients, and any specifically desired result to be achieved. However, in one aspect, the hydrophobic drug may be present in an amount of from about 0.0001% w/w to about 80% w/w of the dispersion, or a pre-concentrate or a composition that is capable of forming such a dispersion. In another aspect, the amount may be from about 1% w/w to about 20% w/w. In an additional aspect, when the drug is cyclosporine, it may be included in an amount of from about 8% w/w to about 15% w/w of the pre-concentrate. In another aspect, the amount of cyclosporine may be from about 10% w/w to about 13% w/w of the pre-concentrate.

In one embodiment of the invention, the active ingredient is completely solubilized in the pre-concentrate that is capable of forming the bioavailable aqueous dispersion upon in contact with an aqueous medium. In another embodiment, the active ingredient is at least partially solubilized in the pre-concentrate.

In some embodiments of the invention, the dispersion may include a second drug in addition to the original hydrophobic active agent. Such a drug may be either hydrophobic or hydrophilic (i.e. having an aqueous solubility of greater than about 1 mg/ml at about 25° C., in a completely solubilized, suspended, or partially solubilized and partially suspended form). In one aspect, such a second drug may be a macrolide, such as sirolimus, or tacrolimus.

B. Solubilizers

A wide variety of agents may be used as solubilizers for the present invention. In general, the solubilizer is a solvent, or a mixture of solvents that are capable of at least partially solubilizing the hydrophobic therapeutic agent when presented in an effective amount. In one aspect, the solubilizer may be present in an amount sufficient to completely solubilize the hydrophobic therapeutic agent. Examples of suitable solubilizers may include without limitation, alcohols and polyols, such as ethanol, isopropanol, butanol, benzyl alcohol, ethylene glycol, propylene glycol, butanediols and isomers thereof, glycerol, diglycerol, polyglycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclo dextrin derivatives. Additional examples include without limitation, ethers of polyethylene glycols having an average molecular weight of about 200 to about 6000, such as tetrahydrofurfuryl alcohol PEG ether (glycofurol, available commercially from BASF under the trade name Tetraglycol) or methoxy PEG (Union Carbide). Further examples may include without limitation, amides, such as 2-pyrrolidone, 2-piperidone, ε-caprolactam, N-alkylpyrrolidone, N-hydroxyalkylpyrrolidone, N-alkylpiperidone, N-alkylcaprolactam, dimethyl acetamide, and polyvinylpyrrolidone, as well as esters, such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, Δ-valerolactone and isomers thereof, β-butyrolactone and isomers thereof. Other solubilizers known in the art, may also be used, such as dimethyl acetamide, dimethyl isosorbide (Arlasolve DMI (ICI)), N-methylpyrrolidones (Pharmnasolve (ISP)), monooctanoin, diethylene glycol monoethyl ether (available from Gattefosse under the trade name Transcutol), and water. Moreover, mixtures of solubilizers are within the scope of the present invention.

Of course, the selection of specific amounts and types of solubilizer may depend on various criteria, including the specific pharmaceutically active agent being employed, as well as specifically desired results to be achieved. However, in one aspect, when the hydrophobic drug is cyclosporine, the solubilizer may be either ethanol, propylene glycol, or a mixture thereof.

The amount of the solubilizer should generally be an amount that is sufficient to at least partially, and in some aspects, to completely solubilize the hydrophobic drug in a composition which is capable of creating the aqueous dispersion of the present invention. Moreover, additional amounts of solubilizer may be included in order to compensate for certain dosage formulations, such as oral dosage gelatin capsules which absorb a significant amount of water during the fabrication and storage thereof. Additionally, when the solubilizer is a volatile solvent, the total amount of solubilizer included may be adjusted to compensate for loss due to evaporation during the manufacturing process and storage. In one aspect, the amount of solubilizer used may be from about 0.0001% w/w to about 99% w/w of the dispersion or a composition capable of creating such a dispersion. In another aspect, the amount of solubilizer may be from about 1% w/w to about 90% w/w. In another aspect, the amount of solubilizer may be from about 5% w/w to about 20% w/w. In yet another aspect, the amount may be from about 7% w/w to about 16% w/w.

As noted above, in some instances the hydrophobic drug may be only partially solubilized in the dispersion. In one aspect, they hydrophobic drug may be partially solubilized by having the solubilizer present in an amount of from about 10% w/w to about 50% w/w of the hydrophobic active agent. In another aspect the amount may be about 10% w/w. In yet another aspect, the amount may be about 30% w/w. In yet another aspect, the amount may be about 50% w/w. The amount of solubilizer may also be recited in terms of ratios of solubilizer to hydrophobic active agent. For example, when cyclosporine is used, the amount of solubilizer may be present in a w/w ratio of solubilizer to cyclosporine of greater than about 0.7:1. In another aspect, the ratio of solubilizer to cyclosporine may be greater than about 1:1.

C. Stabilizers

In addition to the hydrophobic active agent and the solubilizer as recited above, the dispersions, and compositions capable of creating such dispersions, of the present invention typically include one or more stabilizers. The stabilizer serves the function of preventing particles of undissolved hydrophobic drug contained in the dispersion from agglomerating for a time sufficient for the drug to be absorbed from the dispersion.

A wide variety of stabilizers can be used in the dispersions and compositions capable of creating such dispersions, of the present invention. However, in one aspect, the stabilizer may be a surfactant. The choice of specific lipophilic and hydrophilic surfactants should be made keeping in mind the particular therapeutic agent to be used in the composition, and the range of polarity appropriate for the chosen therapeutic agent. With these general principles in mind, a very broad range of surfactants is suitable for use in the present invention. Such surfactants can be grouped into the following general chemical classes detailed in the Tables below. The HLB values given in the Tables below generally represent the HLB value as reported by the manufacturer of the corresponding commercial product. In cases where more than one commercial product is listed, the HLB value in the Tables is the value as reported for one of the commercial products, a rough average of the reported values, or a value that, in the judgment of the Applicants, is more reliable. It should be emphasized that the invention is not limited to the surfactants in the following Tables, and which show representative, but not exclusive, lists of available surfactants. Moreover, those of ordinary skill in the art will recognize that not each and every surfactant listed below will work equally as well as a stabilizer for a given drug, but that some will work better than others.

1. Polyethoxylated Fatty Acids

Although polyethylene glycol (PEG) itself does not function as a surfactant, a variety of PEG-fatty acid esters do. Among the PEG-fatty acid monoesters, esters of lauric acid, oleic acid, and stearic acid are most useful. Among the surfactants of Table 1, preferred hydrophilic surfactants include PEG-8 laurate, PEG-8 oleate, PEG-8 stearate, PEG-9 oleate, PEG-10 laurate, PEG-10 oleate, PEG-12 laurate, PEG-12 oleate, PEG-15 oleate, PEG-20 laurate and PEG-20 oleate. Examples of polyethoxylated fatty acid monoester surfactants commercially available are shown in Table 1.

TABLE 1
PEG-Fatty Acid Monoester Surfactants

| COMPOUND | COMMERCIAL PRODUCT (Supplier) | HLB |
|---|---|---|
| PEG 4-100 monolaurate | Crodet L series (Croda) | >9 |
| PEG 4-100 monooleate | Crodet O series (Croda) | >8 |
| PEG 4-100 monostearate | Crodet S series (Croda), Myrj Series | >6 |
| | (Atlas/ICI) | |
| PEG 400 distearate | Cithrol 4DS series (Croda) | >10 |
| PEG 100, 200, 300 monolaurate | Cithrol ML series (Croda) | >10 |
| PEG 100, 200, 300 monooleate | Cithrol MO series (Croda) | >10 |
| PEG 400 dioleate | Cithrol 4DO series (Croda) | >10 |
| PEG 400-1000 monostearate | Cithrol MS series (Croda) | >10 |
| PEG-1 stearate | Nikkol MYS-1EX (Nikko), Coster K1 (Condea) | 2 |
| PEG-2 stearate | Nikkol MYS-2 (Nikko) | 4 |
| PEG-2 oleate | Nikkol MYO-2 (Nikko) | 4.5 |
| PEG-4 laurate | Mapeg ® 200 ML (PPG), Kessco ® PEG | 9.3 |
| | 200 ML (Stepan), LIPOPEG 2 L (LIPO Chem.) | |
| PEG-4 oleate | Mapeg ® 200 MO (PPG), Kessco ® PEG | 8.3 |
| | 200 MO (Stepan) | |
| PEG-4 stearate | Kessco ® PEG 200 MS (Stepan), Hodag | 6.5 |
| | 20 S (Calgene), Nikkol MYS-4 (Nikko) | |
| PEG-5 stearate | Nikkol TMGS-5 (Nikko) | 9.5 |
| PEG-5 oleate | Nikkol TMGO-5 (Nikko) | 9.5 |
| PEG-6 oleate | Algon OL 60 (Auschem SpA), Kessco ® | 8.5 |
| | PEG 300 MO (Stepan), Nikkol MYO-6 | |
| | (Nikko), Emulgante A6 (Condea) | |
| PEG-7 oleate | Algon OL 70 (Auschem SpA) | 10.4 |
| PEG-6 laurate | Kessco ® PEG300 ML (Stepan) | 11.4 |
| PEG-7 laurate | Lauridac 7 (Condea) | 13 |
| PEG-6 stearate | Kessco ® PEG300 MS (Stepan) | 9.7 |
| PEG-8 laurate | Mapeg ® 400 ML (PPG), | 13 |
| | LIPOPEG 4DL(Lipo Chem.) | |
| PEG-8 oleate | Mapeg ® 400 MO (PPG), Emulgante A8 (Condea) | 12 |
| PEG-8 stearate | Mapeg ® 400 MS (PPG), Myrj 45 | 12 |
| PEG-9 oleate | Emulgante A9 (Condea) | >10 |
| PEG-9 stearate | Cremophor S9 (BASF) | >10 |
| PEG-10 laurate | Nikkol MYL-10 (Nikko), Lauridac 10 (Croda) | 13 |
| PEG-10 oleate | Nikkol MYO-10 (Nikko) | 11 |
| PEG-12 stearate | Nikkol MYS-10 (Nikko), Coster K100 (Condea) | 11 |
| PEG-12 laurate | Kessco ® PEG 600 ML (Stepan) | 15 |
| PEG-12 oleate | Kessco ® PEG 600 MO (Stepan) | 14 |
| PEG-12 ricinoleate | (CAS # 9004-97-1) | >10 |
| PEG-12 stearate | Mapeg ® 600 MS (PPG), Kessco ® PEG | 14 |
| | 600 MS (Stepan) | |
| PEG-15 stearate | Nikkol TMGS-15 (Nikko), Koster K15 (Condea) | 14 |
| PEG-15 oleate | Nikkol TMGO-15 (Nikko) | 15 |
| PEG-20 laurate | Kessco ® PEG 1000 ML (Stepan) | 17 |
| PEG-20 oleate | Kessco ® PEG 1000 MO (Stepan) | 15 |
| PEG-20 stearate | Mapeg ® 1000 MS (PPG), Kessco ® | 16 |
| | PEG 1000 MS (Stepan), Myrj 49 | |
| PEG-25 stearate | Nikkol MYS-25 (Nikko) | 15 |
| PEG-32 laurate | Kessco ® PEG 1540 ML (Stepan) | 16 |
| PEG-32 oleate | Kessco ® PEG 1540 MO (Stepan) | 17 |
| PEG-32 stearate | Kessco ® PEG 1540 MS (Stepan) | 17 |
| PEG-30 stearate | Myrj 51 | >10 |
| PEG-40 laurate | Crodet L40 (Croda) | 17.9 |
| PEG-40 oleate | Crodet O40 (Croda) | 17.4 |
| PEG-40 stearate | Myrj 52, Emerest ® 2715 (Henkel), | >10 |
| | Nikkol MYS-40 (Nikko) | |
| PEG-45 stearate | Nikkol MYS-45 (Nikko) | 18 |
| PEG-50 stearate | Myrj 53 | >10 |
| PEG-55 stearate | Nikkol MYS-55 (Nikko) | 18 |
| PEG-100 oleate | Crodet O-100 (Croda) | 18.8 |
| PEG-100 stearate | Myrj 59, Ariacel 165 (ICI) | 19 |
| PEG-200 oleate | Albunol 200 MO (Taiwan Surf.) | >10 |
| PEG-400 oleate | LACTOMUL (Henkel), Albunol 400 | >10 |
| | MO (Taiwan Surf.) | |
| PEG-600 oleate | Albunol 600 MO (Taiwan Surf.) | >10 |

2. PEG-Fatty Acid Diesters

Polyethylene glycol fatty acid diesters are also suitable for use as surfactants in the compositions of the present invention. Among the surfactants in Table 2, preferred hydrophilic surfactants include PEG-20 dilaurate, PEG-20 dioleate, PEG-20 distearate, PEG-32 dilaurate and PEG-32 dioleate. Representative PEG-fatty acid diesters are shown in Table 2.

TABLE 2
PEG-Fatty Acid Diester Surfactants

| COMPOUND | COMMERCIAL PRODUCT (Supplier) | HLB |
|---|---|---|
| PEG-4 dilaurate | Mapeg ® 200 DL (PPG), Kessco ® PEG | 7 |
| | 200 DL (Stepan), LIPOPEG 2-DL | |
| | (Lipo Chem.) | |
| PEG-4 dioleate | Mapeg ® 200 DO (PPG), | 6 |
| PEG-4 distearate | Kessco ® 200 DS (Stepan) | 5 |
| PEG-6 dilaurate | Kessco ® PEG 300 DL (Stepan) | 9.8 |
| PEG-6 dioleate | Kessco ® PEG 300 DO (Stepan) | 7.2 |
| PEG-6 distearate | Kessco ® PEG 300 DS (Stepan) | 6.5 |
| PEG-8 dilaurate | Mapeg ® 400 DL (PPG), Kessco ® PEG | 11 |
| | 400 DL (Stepan), LIPOPEG 4 DL | |
| | (Lipo Chem.) | |
| PEG-8 dioleate | Mapeg ® 400 DO (PPG), Kessco ® PEG | 8.8 |
| | 400 DO (Stepan), LIPOPEG 4 DO | |
| | (Lipo Chem.) | |
| PEG-8 distearate | Mapeg ® 400 DS (PPG), CDS 400 (Nikkol) | 11 |
| PEG-10 dipalmitate | Polyaldo 2PKFG | >10 |
| PEG-12 dilaurate | Kessco ® PEG 600 DL (Stepan) | 11.7 |
| PEG-12 distearate | Kessco ® PEG 600 DS (Stepan) | 10.7 |
| PEG-12 dioleate | Mapeg ® 600 DO (PPG), Kessco ® 600 | 10 |
| | DO (Stepan) | |
| PEG-20 dilaurate | Kessco ® PEG 1000 DL (Stepan) | 15 |
| PEG-20 dioleate | Kessco ® PEG 1000 DO (Stepan) | 13 |
| PEG-20 distearate | Kessco ® PEG 1000 DS (Stepan) | 12 |
| PEG-32 dilaurate | Kessco ® PEG 1540 DL (Stepan) | 16 |
| PEG-32 dioleate | Kessco ® PEG 1540 DO (Stepan) | 15 |
| PEG-32 distearate | Kessco ® PEG 1540 DS (Stepan) | 15 |
| PEG-400 dioleate | Cithrol 4DO series (Croda) | >10 |
| PEG-400 distearate | Cithrol 4DS series (Croda) | >10 |

3. PEG-Fatty Acid Mono- and Di-ester Mixtures

In general, mixtures of surfactants are also useful in the present invention, including mixtures of two or more commercial surfactant products. Several PEG-fatty acid esters are marketed commercially as mixtures or mono- and diesters. Representative surfactant mixtures are shown in Table 3.

TABLE 3
PEG-Fatty Acid Mono- and Diester Mixtures

| | COMMERCIAL | |
|---|---|---|
| COMPOUND | PRODUCT (Supplier) | HLB |
| PEG 4-150 mono, dilaurate | Kessco ® PEG 200-6000 mono, | |
| | dilaurate (Stepan) | |
| PEG 4-150 mono, dioleate | Kessco ® PEG 200-6000 mono, | |
| | dioleate (Stepan) | |
| PEG 4-150 mono, distearate | Kessco ® 200-6000 mono, | |
| | distearate (Stepan) | |

4. Polyethylene Glycol Glycerol Fatty Acid Esters

Suitable PEG glycerol fatty acid esters are shown in Table 5. Among the surfactants in the Table, preferred hydrophilic surfactants are PEG-20 glyceryl laurate, PEG-30 glyceryl laurate, PEG-40 glyceryl laurate, PEG-20 glyceryl oleate, and PEG-30 glyceryl oleate.

TABLE 4
PEG Glycerol Fatty Acid Esters

| | COMMERCIAL PRODUCT | |
|---|---|---|
| COMPOUND | (Supplier) | HLB |
| PEG-20 glyceryl laurate | Tagat ® L (Goldschmidt) | 16 |
| PEG-30 glyceryl laurate | Tagat ® L2 (Goldschmidt) | 16 |
| PEG-15 glyceryl laurate | Glycerox L series (Croda) | 15 |
| PEG-40 glyceryl laurate | Glycerox L series (Croda) | 15 |
| PEG-20 glyceryl stearate | Capmul ® EMG (ABITEC), | 13 |
| | Aldo ® MS-20 KFG (Lonza) | |
| PEG-20 glyceryl oleate | Tagat ® O (Goldschmidt) | >10 |
| PEG-30 glyceryl oleate | Tagat ® O2 (Goldschmidt) | >10 |

5. Alcohol-Oil Transesterification Products

A large number of surfactants of different degrees of lipophilicity or hydrophilicity can be prepared by reaction of alcohols or polyalcohols with a variety of natural and/or hydrogenated oils. Most commonly, the oils used are castor oil or hydrogenated castor oil, or an edible vegetable oil such as corn oil, olive oil, peanut oil, palm kernel oil, apricot kernel oil, or almond oil. Preferred alcohols include glycerol, propylene glycol, ethylene glycol, polyethylene glycol, sorbitol, and pentaerythritol. Among these alcohol-oil transesterified surfactants, preferred hydrophilic surfactants are PEG-35 castor oil (Incrocas-35), PEG-40 hydrogenated castor oil (Cremophor RH 40), PEG-25 trioleate (TAGAT® TO), PEG-60 corn glycerides (Crovol M70), PEG-60 almond oil (Crovol A70), PEG-40 palm kernel oil (Crovol PK70), PEG-50 castor oil (Emalex C-50), PEG-50 hydrogenated castor oil (Emalex HC-50), PEG-8 caprylickcapric glycerides (Labrasol), and PEG-6 caprylic/capric glycerides (Softigen 767). Preferred lipophilic surfactants in this class include PEG-5 hydrogenated castor oil, PEG-7 hydrogenated castor oil, PEG-9 hydrogenated castor oil, PEG-6 corn oil (Labrafil® M 2125 CS), PEG-6 almond oil (Labrafil® M 1966 CS), PEG-6 apricot kernel oil (Labrafil® M 1944 CS), PEG-6 olive oil (Labrafil® M 1980 CS), PEG-6 peanut oil (Labrafil® M 1969 CS), PEG-6 hydrogenated palm kernel oil (Labrafil® M 2130 BS), PEG-6 palm kernel oil (Labrafil® M 2130 CS), PEG-6 triolein (Labrafil® M 2735 CS), PEG-8 corn oil (Labrafil® WL 2609 BS), PEG-20 corn glycerides (Crovol M40), and PEG-20 almond glycerides (Crovol A40). The latter two surfactants are reported to have HLB values of 10, which is generally considered to be the approximate border line between hydrophilic and lipophilic surfactants. For purposes of the present invention, these two surfactants are considered to be lipophilic.

Representative surfactants of this class suitable for use in the present invention are shown in Table 5.

TABLE 5
Transesterification Products of Oils and Alcohols

| COMPOUND | COMMERCIAL PRODUCT (Supplier) | HLB |
|---|---|---|
| PEG-3 castor oil | Nikkol CO-3 (Nikko) | 3 |
| PEG-5, 9, and 16 castor oil | ACCONON CA series (ABITEC) | 6-7 |
| PEG-20 castor oil | Emalex C-20 (Nihon Emulsion), Nikkol CO-20 TX | 11 |
| | (Nikko) | |
| PEG-23 castor oil | Emulgante EL23 | >10 |
| PEG-30 castor oil | Emalex C-30 (Nihon Emulsion), Alkamuls ® EL | 11 |
| | 620 (Rhone-Poulenc), Incrocas 30 (Croda) | |
| PEG-35 castor oil | Cremophor EL and EL-P (BASF), Emulphor EL, | |
| | Incrocas-35 (Croda), Emulgin RO 35 (Henkel) | |
| PEG-38 castor oil | Emulgante EL 65 (Condea) | |
| PEG-40 castor oil | Emalex C-40 (Nihon Emulsion), Alkamuls ® EL | 13 |
| | 719 (Rhone-Poulenc) | |
| PEG-50 castor oil | Emalex C-50 (Nihon Emulsion) | 14 |
| PEG-56 castor oil | Eumulgin ® PRT 56 (Pulcra SA) | >10 |
| PEG-60 castor oil | Nikkol CO-60TX (Nikko) | 14 |
| PEG-100 castor oil | Thornley | >10 |
| PEG-200 castor oil | Eumulgin ® PRT 200 (Pulcra SA) | >10 |
| PEG-5 hydrogenated castor oil | Nikkol HCO-5 (Nikko) | 6 |
| PEG-7 hydrogenated castor oil | Simusol ® 989 (Seppic), Cremophor WO7 | 6 |
| | (BASF) | |
| PEG-10 hydrogenated castor oil | Nikkol HCO-10 (Nikko) | 6.5 |
| PEG-20 hydrogenated castor oil | Nikkol HCO-20 (Nikko) | 11 |
| PEG-25 hydrogenated castor oil | Simulsol ® 1292 (Seppic), Cerex ELS 250 | 11 |
| | (Auschem SpA) | |
| PEG-30 hydrogenated castor oil | Nikkol HCO-30 (Nikko) | 11 |
| PEG-40 hydrogenated castor oil | Cremophor RH 40 (BASF), Croduret (Croda), | 13 |
| | Emulgin HRE 40 | |
| | (Henkel) | |
| PEG-45 hydrogenated castor oil | Cerex ELS 450 (Auschem Spa) | 14 |
| PEG-50 hydrogenated castor oil | Emalex HC-50 (Nihon Emulsion) | 14 |
| PEG-60 hydrogenated castor oil | Nikkol HCO-60 (Nikko); Cremophor RH 60 | 15 |
| | (BASF) | |
| PEG-80 hydrogenated castor oil | Nikkol HCO-80 (Nikko) | 15 |
| PEG-100 hydrogenated castor oil | Nikkol HCO-100 (Nikko) | 17 |
| PEG-6 corn oil | Labrafil ® M 2125 CS (Gattefosse) | 4 |
| PEG-6 almond oil | Labrafil ® M 1966 CS (Gattefosse) | 4 |
| PEG-6 apricot kernel oil | Labrafil ® M 1944 CS (Gattefosse) | 4 |
| PEG-6 olive oil | Labrafil ® M 1980 CS (Gattefosse) | 4 |
| PEG-6 peanut oil | Labrafil ® M 1969 CS (Gattefosse) | 4 |
| PEG-6 hydrogenated palm kernel oil | Labrafil ® M 2130 BS (Gattefosse) | 4 |
| PEG-6 palm kernel oil | Labrafil ® M 2130 CS (Gattefosse) | 4 |
| PEG-6 triolein | Labrafil ® M 2735 CS (Gattefosse) | 4 |
| PEG-8 corn oil | Labrafil ® WL 2609 BS (Gattefosse) | 6-7 |
| PEG-20 corn glycerides | Crovol M40 (Croda) | 10 |
| PEG-20 almond glycerides | Crovol A40 (Croda) | 10 |
| PEG-25 trioleate | TAGAT ® TO (Goldschmidt) | 11 |
| PEG-40 palm kernel oil | Crovol PK-70 | >10 |
| PEG-60 corn glycerides | Crovol M70(Croda) | 15 |
| PEG-60 almond glycerides | Crovol A70 (Croda) | 15 |
| PEG-4 caprylic/capric triglyceride | Labrafac ® Hydro (Gattefosse), | 4-5 |
| PEG-8 caprylic/capric glycerides | Labrasol (Gattefosse), Labrafac CM 10 | >10 |
| | (Gattefosse) | |
| PEG-6 caprylic/capric glycerides | SOFTIGEN ® 767 (Huls), Glycerox 767 (Croda) | 19 |
| Lauroyl macrogol-32 glyceride | GELUCIRE 44/14 (Gattefosse) | 14 |
| Stearoyl macrogol glyceride | GELUCIRE 50/13 (Gattefosse) | 13 |
| Mono, di, tri, tetra esters of vegetable | SorbitoGlyceride (Gattefosse) | <10 |
| oils and sorbitol | | |
| Pentaerythrityl tetraisostearate | Crodamol PTIS (Croda) | <10 |
| Pentaerythrityl distearate | Albunol DS (Taiwan Surf.) | <10 |
| Pentaerythrityl tetraoleate | Liponate PO-4 (Lipo Chem.) | <10 |
| Pentaerythrityl tetrastearate | Liponate PS-4 (Lipo Chem.) | <10 |
| Pentaerythrityl tetracaprylate/ | Liponate PE-810 (Lipo Chem.), Crodamol PTC | <10 |
| tetracaprate | (Croda) | |
| Pentaerythrityl tetraoctanoate | Nikkol Pentarate 408 (Nikko) | |

Also included as oils in this category of surfactants are oil-soluble vitamins, such as vitamins A, D, E, K, etc. Thus, derivatives of these vitamins, such as tocopheryl PEG-1000 succinate (TPGS, available from Eastman), are also suitable surfactants.

6. Polyglycerized Fatty Acids

Polyglycerol esters of fatty acids are also suitable surfactants for the present invention. Among the polyglyceryl fatty acid esters, preferred lipophilic surfactants include polyglyceryl oleate (Plurol Oleique), polyglyceryl-2 dioleate (Nikkol DGDO), and polyglyceryl-10 trioleate. Preferred hydrophilic surfactants include polyglyceryl-10 laurate (Nikkol Decaglyn 1-L), polyglyceryl-10 oleate (Nikkol Decaglyn 1-0), and polyglyceryl-10 mono, dioleate (Caprol® PEG 860). Polyglyceryl polyricinoleates (Polymuls) are also preferred hydrophilic and lipophilic surfactants. Examples of suitable polyglyceryl esters are shown in Table 6.

TABLE 6
Polyglycerized Fatty Acids

| | COMMERCIAL | |
|---|---|---|
| COMPOUND | PRODUCT (Supplier) | HLB |
| Polyglyceryl-2 stearate | Nikkol DGMS (Nikko) | 5-7 |
| Polyglyceryl-2 oleate | Nikkol DGMO (Nikko) | 5-7 |
| Polyglyceryl-2 isostearate | Nikkol DGMIS (Nikko) | 5-7 |
| Polyglyceryl-3 oleate | Caprol ® 3GO (ABITEC), | 6.5 |
| | Drewpol 3-1-O (Stepan) | |
| Polyglyceryl-4 oleate | Nikkol Tetraglyn 1-O (Nikko) | 5-7 |
| Polyglyceryl-4 stearate | Nikkol Tetraglyn 1-S (Nikko) | 5-6 |
| Polyglyceryl-6 oleate | Drewpol 6-1-O (Stepan), Nikkol | 9 |
| | Hexaglyn 1-O (Nikko) | |
| Polyglyceryl-10 laurate | Nikkol Decaglyn 1-L (Nikko) | 15 |
| Polyglyceryl-10 oleate | Nikkol Decaglyn 1-O (Nikko) | 14 |
| Polyglyceryl-10 stearate | Nikkol Decaglyn 1-S (Nikko) | 12 |
| Polyglyceryl-6 ricinoleate | Nikkol Hexaglyn PR-15 (Nikko) | >8 |
| Polyglyceryl-10 linoleate | Nikkol Decaglyn 1-LN (Nikko) | 12 |
| Polyglyceryl-6 pentaoleate | Nikkol Hexaglyn 5-O (Nikko) | <10 |
| Polyglyceryl-3 dioleate | Cremophor GO32 (BASF) | <10 |
| Polyglyceryl-3 distearate | Cremophor GS32 (BASF) | <10 |
| Polyglyceryl-4 pentaoleate | Nikkol Tetraglyn 5-O (Nikko) | <10 |
| Polyglyceryl-6 dioleate | Caprol ® 6G20 (ABITEC); Hodag | 8.5 |
| | PGO-62 (Calgene), PLUROL | |
| | OLEIQUE CC 497 (Gattefosse) | |
| Polyglyceryl-2 dioleate | Nikkol DGDO (Nikko) | 7 |
| Polyglyceryl-10 trioleate | Nikkol Decaglyn 3-O (Nikko) | 7 |
| Polyglyceryl-10 pentaoleate | Nikkol Decaglyn 5-O (Nikko) | 3.5 |
| Polyglyceryl-10 septaoleate | Nikkol Decaglyn 7-O (Nikko) | 3 |
| Polyglyceryl-10 tetraoleate | Caprol ® 10G4O (ABITEC); Hodag | 6.2 |
| | PGO-62 (CALGENE), Drewpol | |
| | 10-4-O (Stepan) | |
| Polyglyceryl-10 | Nikkol Decaglyn 10-IS (Nikko) | <10 |
| decaisostearate | | |
| Polyglyceryl-101 | Drewpol 10-10-O (Stepan), Caprol | 3.5 |
| decaoleate | 10G10O (ABITEC), Nikkol | |
| | Decaglyn 10-O | |
| Polyglyceryl-10 mono, | Caprol ® PGE 860 (ABITEC) | 11 |
| dioleate | | |
| Polyglyceryl | Polymuls (Henkel) | 3-20 |
| polyricinoleate | | |

7. Propylene Glycol Fatty Acid Esters

Esters of propylene glycol and fatty acids are suitable surfactants for use in the present invention. In this surfactant class, preferred lipophilic surfactants include propylene glycol monolaurate (Lauroglycol FCC), propylene glycol ricinoleate (Propymuls), propylene glycol monooleate (Myverol P-O6), propylene glycol dicaprylate/dicaprate (Captex® 200), and propylene glycol dioctanoate (Captex® 800). Examples of surfactants of this class are given in Table 7.

TABLE 7
Propylene Glycol Fatty Acid Esters

| | COMMERCIAL | |
|---|---|---|
| COMPOUND | PRODUCT (Supplier) | HLB |
| Propylene glycol monocaprylate | Capryol 90 (Gattefosse), | <10 |
| | Nikkol Sefsol 218 (Nikko) | |
| Propylene glycol monolaurate | Lauroglycol 90 (Gattefosse), | <10 |
| | Lauroglycol FCC (Gattefosse) | |
| Propylene glycol oleate | Lutrol OP2000 (BASF) | <10 |
| Propylene glycol myristate | Mirpyl | <10 |
| Propylene glycol monostearate | ADM PGME-03 (ADM), LIPO | 3-4 |
| | PGMS (Lipo Chem.), | |
| | Aldo ® PGHMS (Lonza) | |
| Propylene glycol hydroxy | | <10 |
| stearate | | |
| Propylene glycol ricinoleate | PROPYMULS (Henkel) | <10 |
| Propylene glycol isostearate | | <10 |
| Propylene glycol monooleate | Myverol P-O6 (Eastman) | <10 |
| Propylene glycol | Captex ® 200 (ABITEC), | >6 |
| dicaprylate/dicaprate | Miglyol ® 840 | |
| | (Huls), Neobee ® | |
| | M-20 (Stepan) | |
| Propylene glycol dioctanoate | Captex ® 800 (ABITEC) | >6 |
| Propylene glycol | LABRAFAC PG (Gattefosse) | >6 |
| caprylate/caprate | | |
| Propylene glycol dilaurate | | >6 |
| Propylene glycol distearate | Kessco ® PGDS (Stepan) | >6 |
| Propylene glycol dicaprylate | Nikkol Sefsol 228 (Nikko) | >6 |
| Propylene glycol dicaprate | Nikkol PDD (Nikko) | >6 |

8. Mixtures of Propylene Glycol Esters-Glycerol Esters

In general, mixtures of surfactants are also suitable for use in the present invention. In particular, mixtures of propylene glycol fatty acid esters and glycerol fatty acid esters are suitable and are commercially available. One preferred mixture is composed of the oleic acid esters of propylene glycol and glycerol (Arlacel 186). Examples of these surfactants are shown in Table 8.

TABLE 8
Glycerol/Propylene Glycol Fatty Acid Esters

| | COMMERCIAL | |
|---|---|---|
| COMPOUND | PRODUCT (Supplier) | HLB |
| Oleic | ATMOS 300, ARLACEL 186 (ICI) | 3-4 |
| Stearic | ATMOS 150 | 3-4 |

9. Mono- and Diglycerides

A particularly important class of surfactants is the class of mono- and diglycerides. These surfactants are generally lipophilic. Preferred lipophilic surfactants in this class of compounds include glyceryl monooleate (Peceol), glyceryl ricinoleate, glyceryl laurate, glyceryl dilaurate (Capmul® GDL), glyceryl dioleate (Capmul® GDO), glyceryl mono/dioleate (Capmul® GMO-K), glyceryl caprylatelcaprate (Capmul® MCM), caprylic acid mono/diglycerides (Imwitor® 988), and mono- and diacetylated monoglycerides (Myvacet® 9-45). Examples of these surfactants are given in Table 9.

TABLE 9
Mono- and Diglyceride Surfactants

| | COMMERCIAL | |
|---|---|---|
| COMPOUND | PRODUCT (Supplier) | HLB |
| Monopalmitolein (C16:1) | (Larodan) | <10 |
| Monoelaidin (C18:1) | (Larodan) | <10 |
| Monocaproin (C6) | (Larodan) | <10 |
| Monocaprylin | (Larodan) | <10 |
| Monocaprin | (Larodan) | <10 |
| Monolaurin | (Larodan) | <10 |
| Glyceryl monomyristate (C14) | Nikkol MGM (Nikko) | 3-4 |
| Glyceryl monooleate (C18:1) | PECEOL (Gattefosse), Hodag | 3-4 |
| | GMO-D, Nikkol MGO (Nikko) | |
| Glyceryl monooleate | RYLO series (Danisco), | 3-4 |
| | DIMODAN series (Danisco), | |
| | EMULDAN (Danisco), | |
| | ALDO ® | |
| | MO FG (Lonza), Kessco GMO | |
| | (Stepan), MONOMULS ® | |
| | series (Henkel), TEGIN O, | |
| | DREWMULSE GMO (Stepan), | |
| | Atlas G-695 (ICI), GMOrphic | |
| | 80 (Eastman), ADM DMG- | |
| | 40, 70, and 100 (ADM), | |
| | Myverol (Eastman) | |
| Glycerol monooleate/linoleate | OLICINE (Gattefosse) | 3-4 |
| Glycerol monolinoleate | Maisine (Gattefosse), | 3-4 |
| | MYVEROL 18-92, | |
| | Myverol 18-06 (Eastman) | |
| Glyceryl ricinoleate | Softigen ® 701 (Huls), | 6 |
| | HODAG GMR-D (Calgene), | |
| | ALDO ® MR (Lonza) | |
| Glyceryl monolaurate | ALDO ® MLD (Lonza), | 6.8 |
| | Hodag GML (Calgene) | |
| Glycerol monopalmitate | Emalex GMS-P (Nihon) | 4 |
| Glycerol monostearate | Capmul ® GMS (ABITEC), | 5-9 |
| | Myvaplex (Eastman), | |
| | IMWITOR ® 191 (Huls), | |
| | CUTINA GMS, Aldo ® MS | |
| | (Lonza), Nikkol MGS series | |
| | (Nikko) | |
| Glyceryl mono-,dioleate | Capmul ® GMO-K (ABITEC) | <10 |
| Glyceryl palmitic/stearic | CUTINA MD-A, ESTAGEL- | <10 |
| | G18 | |
| Glyceryl acetate | Lamegin ® EE (Grunau GmbH) | <10 |
| Glyceryl laurate | Imwitor ® 312 (Huls), | 4 |
| | Monomuls ® 90-45 (Grunau | |
| | GmbH), Aldo ® MLD (Lonza) | |
| Glyceryl citrate/lactate/ | Imwitor ® 375 (Huls) | <10 |
| oleate/linoleate | | |
| Glyceryl caprylate | Imwitor ® 308 (Huls), | 5-6 |
| | Capmul ® MCMC8 (ABITEC) | |
| Glyceryl caprylate/caprate | Capmul ® MCM (ABITEC) | 5-6 |
| Caprylic acid mono, | Imwitor ® 988 (Huls) | 5-6 |
| diglycerides | | |
| Caprylic/capric glycerides | Imwitor ® 742 (Huls) | <10 |
| Mono-and diacetylated | Myvacet ® 9-45, Myvacet ® | 3.8-4 |
| monoglycerides | 9-40, Myvacet ® 9-08 | |
| | (Eastman), Lamegin ® | |
| | (Grunau) | |
| Glyceryl monostearate | Aldo ® MS, Arlacel 129 (ICI), | 4.4 |
| | LIPO GMS (Lipo Chem.), | |
| | Imwitor ® 191 (Huls), | |
| | Myvaplex (Eastman) | |
| Lactic acid esters of | LAMEGIN GLP (Henkel) | <10 |
| mono,diglycerides | | |
| Dicaproin (C6) | (Larodan) | <10 |
| Dicaprin (C10) | (Larodan) | <10 |
| Dioctanoin (C8) | (Larodan) | <10 |
| Dimyristin (C14) | (Larodan) | <10 |
| Dipalmitin (C16) | (Larodan) | <10 |
| Distearin | (Larodan) | <10 |
| Glyceryl dilaurate (C12) | Capmul ® GDL (ABITEC) | 3-4 |
| Glyceryl dioleate | Capmul ® GDO (ABITEC) | 3-4 |
| Glycerol esters of fatty acids | GELUCIRE 39/01 (Gattefosse), | 1 |
| | GELUCIRE 43/01 (Gattefosse) | 6 |
| | GELUCIRE 37/06 (Gattefosse) | |
| Dipalmitolein (C16:1) | (Larodan) | <10 |
| 1,2 and 1,3-diolein (C18:1) | (Larodan) | <10 |
| Dielaidin (C18:1) | (Larodan) | <10 |
| Dilinolein (C18:2) | (Larodan) | <10 |

10. Sterol and Sterol Derivatives

Sterols and derivatives of sterols are suitable surfactants for use in the present invention. These surfactants can be hydrophilic or lipophilic. Preferred derivatives include the polyethylene glycol derivatives. A preferred lipophilic surfactant in this class is cholesterol. A preferred hydrophilic surfactant in this class is PEG-24 cholesterol ether Solulan C-24). Examples of surfactants of this class are shown in Table 10.

TABLE 10
Sterol and Sterol Derivative Surfactants

| | COMMERCIAL PRODUCT | |
|---|---|---|
| COMPOUND | (Supplier) | HLB |
| Cholesterol, sitosterol, lanosterol | | <10 |
| PEG-24 cholesterol ether | Solulan C-24 (Amerchol) | >10 |
| PEG-30 cholestanol | Nikkol DHC (Nikko) | >10 |
| Phytosterol | GENEROL series (Henkel) | <10 |
| PEG-25 phyto sterol | Nikkol BPSH-25 (Nikko) | >10 |
| PEG-5 soya sterol | Nikkol BPS-5 (Nikko) | <10 |
| PEG-10 soya sterol | Nikkol BPS-10 (Nikko) | <10 |
| PEG-20 soya sterol | Nikkol BPS-20 (Nikko) | <10 |
| PEG-30 soya sterol | Nikkol BPS-30 (Nikko) | >10 |

11. Polyethylene Glycol Sorbitan Fatty Acid Esters

A variety of PEG-sorbitan fatty acid esters are available and are suitable for use as surfactants in the present invention. In general, these surfactants are hydrophilic, although several lipophilic surfactants of this class can be used. Among the PEG-sorbitan fatty acid esters, preferred hydrophilic surfactants include PEG-20 sorbitan monolaurate (Tween-20), PEG-20 sorbitan monopalmitate (Tween-40), PEG-20 sorbitan monostearate (Tween-60), and PEG-20 sorbitan monooleate (Tween-80). Examples of these surfactants are shown in Table 11.

TABLE 11
PEG-Sorbitan Fatty Acid Esters

| | COMMERCIAL | |
|---|---|---|
| COMPOUND | PRODUCT (Supplier) | HLB |
| PEG-10 sorbitan laurate | Liposorb L-10 (Lipo Chem.) | >10 |
| PEG-20 sorbitan monolaurate | Tween-20 (Atlas/ICI), Crillet 1 | 17 |
| | (Croda), DACOL MLS 20 | |
| | (Condea) | |
| PEG-4 sorbitan monolaurate | Tween-21 (Atlas/ICI), Crillet | 13 |
| | 11 (Croda) | |
| PEG-80 sorbitan monolaurate | Hodag PSML-80 (Calgene); | >10 |
| | T-Maz 28 | |
| PEG-6 sorbitan monolaurate | Nikkol GL-1 (Nikko) | 16 |
| PEG-20 sorbitan | Tween-40 (Atlas/ICI), Crillet 2 | 16 |
| monopalmitate | (Croda) | |
| PEG-20 sorbitan monostearate | Tween-60 (Atlas/ICI), Crillet 3 | 15 |
| | (Croda) | |
| PEG-4 sorbitan monostearate | Tween-61 (Atlas/ICI), Crillet 31 | 9.6 |
| | (Croda) | |
| PEG-8 sorbitan monostearate | DACOL MSS (Condea) | >10 |
| PEG-6 sorbitan monostearate | Nikkol TS106 (Nikko) | 11 |
| PEG-20 sorbitan tristearate | Tween-65 (Atlas/ICI), Crillet 35 | 11 |
| | (Croda) | |
| PEG-6 sorbitan tetrastearate | Nikkol GS-6 (Nikko) | 3 |
| PEG-60 sorbitan tetrastearate | Nikkol GS-460 (Nikko) | 13 |
| PEG-5 sorbitan monooleate | Tween-81 (Atlas/ICI), Crillet 41 | 10 |
| | (Croda) | |
| PEG-6 sorbitan monooleate | Nikkol TO-106 (Nikko) | 10 |
| PEG-20 sorbitan monooleate | Tween-80 (Atlas/ICI), Crillet 4 | 15 |
| | (Croda) | |
| PEG-40 sorbitan oleate | Emalex ET 8040 (Nihon | 18 |
| | Emulsion) | |
| PBG-20 sorbitan trioleate | Tween-85 (Atlas/ICI), Crillet 45 | 11 |
| | (Croda) | |
| PEG-6 sorbitan tetraoleate | Nikkol GO-4 (Nikko) | 8.5 |
| PEG-30 sorbitan tetraoleate | Nikkol GO-430 (Nikko) | 12 |
| PEG-40 sorbitan tetraoleate | Nikkol GO-440 (Nikko) | 13 |
| PEG-20 sorbitan | Tween-120 (Atlas/ICI), Crillet 6 | >10 |
| monoisostearate | (Croda) | |
| PEG sorbitol hexaoleate | Atlas G-1086 (ICI) | 10 |
| PEG-6 sorbitol hexastearate | Nikkol GS-6 (Nikko) | 3 |

12. Polyethylene Glycol Alkyl Ethers

Ethers of polyethylene glycol and alkyl alcohols are suitable surfactants for use in the present invention. Preferred lipophilic ethers include PEG-3 oleyl ether (Volpo 3) and PEG-4 lauryl ether (Brij 30). Examples of these surfactants are shown in Table 12.

TABLE 12
Polyethylene Glycol Alkyl Ethers

| | COMMERCIAL | |
|---|---|---|
| COMPOUND | PRODUCT (Supplier) | HLB |
| PEG-2 oleyl ether,oleth-2 | Brij 92/93 (Atlas/ICI) | 4.9 |
| PEG-3 oleyl ether,oleth-3 | Volpo 3 (Croda) | <10 |
| PEG-5 oleyl ether,oleth-5 | Volpo 5 (Croda) | <10 |
| PEG-10 oleyl ether,oleth-10 | Volpo 10 (Croda), Brij 96/97 | 12 |
| | (Atlas/ICI) | |
| PEG-20 oleyl ether,oleth-20 | Volpo 20 (Croda), Brij 98/99 | 15 |
| | (Atlas/ICI) | |
| PEG-4 lauryl ether, laureth-4 | Brij 30 (Atlas/ICI) | 9.7 |
| PEG-9 lauryl ether | | >10 |
| PEG-23 lauryl ether, laureth-23 | Brij 35 (Atlas/ICI) | 17 |
| PEG-2 cetyl ether | Brij 52 (ICI) | 5.3 |
| PEG-10 cetyl ether | Brij 56 (ICI) | 13 |
| PEG-20 cetyl ether | BriJ 58 (ICI) | 16 |
| PEG-2 stearyl ether | Brij 72 (ICI) | 4.9 |
| PEG-10 stearyl ether | Brij 76 (ICI) | 12 |
| PEG-20 stearyl ether | Brij 78 (ICI) | 15 |
| PEG-100 stearyl ether | Brij 700 (ICI) | >10 |

13. Sugar Esters

Esters of sugars are suitable surfactants for use in the present invention. Preferred hydrophilic surfactants in this class include sucrose monopalmitate and sucrose monolaurate. Examples of such surfactants are shown in Table 13.

TABLE 13
Sugar Ester Surfactants

| | COMMERCIAL PRODUCT | |
|---|---|---|
| COMPOUND | (Supplier) | HLB |
| Sucrose distearate | SUCRO ESTER 7 (Gattefosse), | 3 |
| | Crodesta F-10 (Croda) | |
| Sucrose distearate/monostearate | SUCRO ESTER 11 (Gattefosse), | 12 |
| | Crodesta F-110 (Croda) | |
| Sucrose dipalmitate | | 7.4 |
| Sucrose monostearate | Crodesta F-160 (Croda) | 15 |
| Sucrose monopalmitate | SUCRO ESTER 15 (Gattefosse) | >10 |
| Sucrose monolaurate | Saccharose monolaurate 1695 | 15 |
| | (Mitsubisbi-Kasei) | |

14. Polyethylene Glycol Alkyl Phenols

Several hydrophilic PEG-alkyl phenol surfactants are available, and are suitable for use in the present invention. Examples of these surfactants are shown in Table 14.

TABLE 14
Polyethylene Glycol Alkyl Phenol Surfactants

| | COMMERCIAL PRODUCT | |
|---|---|---|
| COMPOUND | (Supplier) | HLB |
| PEG-10-100 | Triton X series (Rohm & Haas), Igepal CA | >10 |
| nonyl phenol | series (GAF, USA), Antarox CA series | |
| | (GAF, UK) | |
| PEG-15-100 | Triton N-series (Rohm & Haas), Igepal CO | >10 |
| octyl phenol | series (GAF, USA), Antarox CO series | |
| ether | (GAF, UK) | |

15. Polyoxyethylene-Polyoxypropylene Block Copolymers

The POE-POP block copolymers are a unique class of polymeric surfactants. The unique structure of the surfactants, with hydrophilic POE and lipophilic POP moieties in well-defined ratios and positions, provides a wide variety of surfactants suitable for use in the present invention. These surfactants are available under various trade names, including Synperonic PE series (ICI); Pluronic® series (BASF), Emkalyx, Lutrol (BASF), Supronic, Monolan, Pluracare, and Plurodac. The generic term for these polymers is “poloxamer” (CAS 9003-11-6). These polymers have the formula:

HO(C<2>H<4>O)<a>(C<3>H<6>O)<b>(C<2>H<4>O)<a>H

where “a” and “b” denote the number of polyoxyethylene and polyoxypropylene units, respectively.

Preferred hydrophilic surfactants of this class include Poloxamers 108, 188, 217, 238, 288, 338, and 407. Preferred lipophilic surfactants in this class include Poloxamers 124, 182, 183, 212, 331, and 335.

Examples of suitable surfactants of this class are shown in Table 15. Since the compounds are widely available, commercial sources are not listed in the Table. The compounds are listed by generic name, with the corresponding “a” and “b” values.

TABLE 15
POE-POP Block Copolymers

| | a, b values in | | |
|---|---|---|---|
| | HO(C<2>H<4>O)<a> | | |
| | (C<3>H<6>O)<b>(C<2>H<4> | | |
| COMPOUND | O)<a>H | | HLB |
| Poloxamer 105 | a = 11 | b = 16 | 8 |
| Poloxamer 108 | a = 46 | b = 16 | >10 |
| Poloxamer 122 | a = 5 | b = 21 | 3 |
| Poloxamer 123 | a = 7 | b = 21 | 7 |
| Poloxamer 124 | a = 11 | b = 21 | >7 |
| Poloxamer 181 | a = 3 | b = 30 | |
| Poloxamer 182 | a = 8 | b = 30 | 2 |
| Poloxamer 183 | a = 10 | b = 30 | |
| Poloxamer 184 | a = 13 | b = 30 | |
| Poloxamer 185 | a = 19 | b = 30 | |
| Poloxamer 188 | a = 75 | b = 30 | 29 |
| Poloxamer 212 | a = 8 | b = 35 | |
| Poloxamer 215 | a = 24 | b = 35 | |
| Poloxamer 217 | a = 52 | b = 35 | |
| Poloxamer 231 | a = 16 | b = 39 | |
| Poloxamer 234 | a = 22 | b = 39 | |
| Poloxamer 235 | a = 27 | b = 39 | |
| Poloxamer 237 | a = 62 | b = 39 | 24 |
| Poloxamer 238 | a = 97 | b = 39 | |
| Poloxamer 282 | a = 10 | b = 47 | |
| Poloxamer 284 | a = 21 | b = 47 | |
| Poloxamer 288 | a = 122 | b = 47 | >10 |
| Poloxamer 331 | a = 7 | b = 54 | 0.5 |
| Poloxamer 333 | a = 20 | b = 54 | |
| Poloxamer 334 | a = 31 | b = 54 | |
| Poloxamer 335 | a = 38 | b = 54 | |
| Poloxamer 338 | a = 128 | b = 54 | |
| Poloxamer 401 | a = 6 | b = 67 | |
| Poloxamer 402 | a = 13 | b = 67 | |
| Poloxamer 403 | a = 21 | b = 67 | |
| Poloxamer 407 | a = 98 | b = 67 | |

16. Sorbitan Fatty Acid Esters

Sorbitan esters of fatty acids are suitable surfactants for use in the present invention. Among these esters, preferred lipophilic surfactants include sorbitan monolaurate (Arlacel 20), sorbitan monopalmitate (Span-40), sorbitan monooleate (Span-80), sorbitan monostearate, and sorbitan tristearate. Examples of these surfactants are shown in Table 16.

TABLE 16
Sorbitan Fatty Acid Ester Surfactants

| | COMMERCIAL PRODUCT | |
|---|---|---|
| COMPOUND | (Supplier) | HLB |
| Sorbitan monolaurate | Span-20 (Atlas/ICI), Crill 1 (Croda), | 8.6 |
| | Arlacel 20 (ICI) | |
| Sorbitan monopalmitate | Span-40 (Atlas/ICI), Crill 2 (Croda), | 6.7 |
| | Nikkol SP-10 (Nikko) | |
| Sorbitan monooleate | Span-80 (Atlas/ICI), Crill 4 (Croda), | 4.3 |
| | Crill 50 (Croda) | |
| Sorbitan monostearate | Span-60 (Atlas/ICI), Crill 3 (Croda), | 4.7 |
| | Nikkol SS-10 (Nikko) | |
| Sorbitan trioleate | Span-85 (Atlas/ICI), Crill 45 (Croda), | 4.3 |
| | Nikkol SO-30 (Nikko) | |
| Sorbitan sesquioleate | Arlacel-C (ICI), Crill 43 (Croda), | 3.7 |
| | Nikkol SO-15 (Nikko) | |
| Sorbitan tristearate | Span-65 (Atlas/ICI) Crill 35 (Croda), | 2.1 |
| | Nikkol SS-30 (Nikko) | |
| Sorbitan monoisostearate | Crill 6 (Croda), Nikkol SI-10 (Nikko) | 4.7 |
| Sorbitan sesquistearate | Nikkol SS-15 (Nikko) | 4.2 |

17. Lower Alcohol Fatty Acid Esters

Esters of lower alcohols (C<2> to C<4>) and fatty acids (C<8> to C<18>) are suitable surfactants for use in the present invention. Among these esters, preferred lipophilic surfactants include ethyl oleate (Crodamol EO), isopropyl myristate (Crodamol IPM), and isopropyl palmitate (Crodamol IPP). Examples of these surfactants are shown in Table 17.

TABLE 17
Lower Alcohol Fatty Acid Ester Surfactants

| | COMMERCIAL PRODUCT | |
|---|---|---|
| COMPOUND | (Supplier) | HLB |
| Ethyl oleate | Crodamol EO (Croda), Nikkol EOO (Nikko) | <10 |
| Isopropyl myristate | Crodamol IPM (Croda) | <10 |
| Isopropyl palmitate | Crodamol IPP (Croda) | <10 |
| Ethyl linoleate | Nikkol VF-E (Nikko) | <10 |
| Isopropyl linoleate | Nikkol VF-IP (Nikko) | <10 |

18. Ionic Surfactants

Ionic surfactants, including cationic, anionic and zwitterionic surfactants, are suitable hydrophilic surfactants for use in the present invention. Preferred anionic surfactants include fatty acid salts and bile salts. Specifically, preferred ionic surfactants include sodium oleate, sodium lauryl sulfate, sodium lauryl sarcosinate, sodium dioctyl sulfosuccinate, sodium cholate, and sodium taurocholate. Examples of such surfactants are shown in Table 18 below. For simplicity, typical counterions are shown in the entries in the Table. It will be appreciated by one skilled in the art, however, that any bioacceptable counterion may be used. For example, although the fatty acids are shown as sodium salts, other cation counterions can also be used, such as alkali metal cations or ammonium. Unlike typical non-ionic surfactants, these ionic surfactants are generally available as pure compounds, rather than commercial (proprietary) mixtures. Because these compounds are readily available from a variety of commercial suppliers, such as Aldrich, Sigma, and the like, commercial sources are not generally listed in Table 18.

TABLE 18
Ionic Surfactants

| COMPOUND | HLB |
|---|---|
| FATTY ACID SALTS | >10 |
| Sodium caproate | |
| Sodium caprylate | |
| Sodium caprate | |
| Sodium laurate | |
| Sodium myristate | |
| Sodium myristolate | |
| Sodium palmitate | |
| Sodium palmitoleate | |
| Sodium oleate | 18 |
| Sodium ricinoleate | |
| Sodium linoleate | |
| Sodium linolenate | |
| Sodium stearate | |
| Sodium lauryl sulfate (dodecyl) | 40 |
| Sodium tetradecyl sulfate | |
| Sodium lauryl sarcosinate | |
| Sodium dioctyl sulfosuccinate [sodium docusate (Cytec)] | |
| BILE SALTS | >10 |
| Sodium cholate | |
| Sodium taurocholate | |
| Sodium glycocholate | |
| Sodium deoxycholate | |
| Sodium taurodeoxycholate | |
| Sodium glycodeoxycholate | |
| Sodium ursodeoxycholate | |
| Sodium chenodeoxycholate | |
| Sodium taurochenodeoxycholate | |
| Sodium glyco cheno deoxycholate | |
| Sodium cholylsarcosinate | |
| Sodium N-methyl taurocholate | |
| PHOSPHOLIPIDS | |
| Egg/Soy lecithin [EpikuronTM (Lucas Meyer), | |
| OvothinTM] (Lucas Meyer)] | |
| Lyso egg/soy lecithin | |
| Hydroxylated lecithin | |
| Lysophosphatidylcholine | |
| Cardiolipin | |
| Sphingomyelin | |
| Phosphatidylcholine | |
| Phosphatidyl ethanolamine | |
| Phosphatidic acid | |
| Phosphatidyl glycerol | |
| Phosphatidyl serine | |
| PHOSPHORIC ACID ESTERS | |
| Diethanolammonium polyoxyethylene-10 oleyl ether phosphate | |
| Esterification products of fatty alcohols or fatty alcohol ethoxylates | |
| with phosphoric acid or anhydride | |
| CARBOXYLATES | |
| Ether carboxylates (by oxidation of terminal OH group of | |
| fatty alcohol ethoxylates) | |
| Succinylated monoglycerides [LAMEGIN ZB (Henkel)] | |
| Sodium stearyl fumarate | |
| Stearoyl propylene glycol hydrogen succinate | |
| Mono/diacetylated tartaric acid esters of mono- and diglycerides | |
| Citric acid esters of mono-, diglycerides | |
| Glyceryl-lacto esters of fatty acids (CFR ref. 172.852) | |
| Acyl lactylates: | |
| lactylic esters of fatty acids | |
| calcium/sodium stearoyl-2-lactylate | |
| calcium/sodium stearoyl lactylate | |
| Alginate salts | |
| Propylene glycol alginate | |
| SULFATES AND SULFONATES | |
| Ethoxylated alkyl sulfates | |
| Alkyl benzene sulfones | |
| α-olefin sulfonates | |
| Acyl isethionates | |
| Acyl taurates | |
| Alkyl glyceryl ether sulfonates | |
| Octyl sulfosuccinate disodium | |
| Disodium undecylenamideo-MEA-sulfosuccinate | |
| CATIONIC Surfactants | >10 |
| Hexadecyl triammonium bromide | |
| Decyl trimethyl ammonium bromide | |
| Cetyl trimethyl ammonium bromide | |
| Dodecyl ammonium chloride | |
| Alkyl benzyldimethylammonium salts | |
| Diisobutyl phenoxyethoxydimethyl benzylammonium salts | |
| Alkylpyridinium salts | |
| Betaines (trialkylglycine) | |
| Lauryl betaine (N-lauryl,N,N-dimethylglycine) | |
| Ethoxylated amines: | |
| Polyoxyethylene-15 coconut amine | |

In one aspect, when the hydrophobic drug is cyclosporine, the stabilizer may be a polyethoxylated surfactant. Many polyethoxylated surfactants are known to those skilled in the art, and may be obtained either synthetically, or by reacting naturally obtained starting materials. Examples of specific polyethoxylated surfactants include without limitation, polyethoxylated castor oils, polyethoxylated hydrogenated castor oils, macrogol glycerides, and mixtures thereof. In one aspect, the polyethoxylated castor oil may be polyoxyl 35 castor oil. In another aspect, the polyethoxylated castor oil may be a hydrogenated castor oil, such as polyoxyl 40 hydrogenated castor oil. Such compounds are commercially available under the respective trade names of CREMOPHOR EL and CREMOPHOR RH40.

In another aspect, the polyethoxylated surfactant may be a macrogol glycerides. Examples of suitable specific macrogol glycerides include without limitation, lauryl macrogol-32 glycerides, stearoyl macrogol-32 glycerides, caprylocaproyl macrogol-8 glycerides, linoleoyl macrogol-6 glycerides, and mixtures thereof. Such compounds are readily commercially available under the respective trade names of GELUCIRE 44/14, GELUCIRE 50/13, LABRASOL, AND LABRAFIL. In one aspect, the macrogol glycerides may be a lauryl macrogol-32 glycerides.

The above recited stabilizer compounds may be used in connection with cyclosporine individually, or in some embodiments may be used in combination. In one aspect, the stabilizer may include a mixture of polyoxyl 35 castor oil and polyoxyl 40 hydrogenated castor oil. In another aspect, such a stabilizer mixture may contain each ingredient in a ratio of from about 3:1 to about 1:3. In yet another aspect, the ratio may be from about 2:1 to about 1:2. In yet another aspect, the ratio may be about 1:1.

As noted above, the specific amount of stabilizer used in the present invention may be varied depending on the specific hydrophobic drug used, and the other ingredients to be included. However, in one aspect of the invention, the stabilizer may be present in an amount of from about 0.01% w/w to about 99% w/w of the dispersion or composition capable of creating such a dispersion. In another aspect, the amount may be from about 1% w/w to about 95% w/w. In another aspect, the amount may be from about 10% w/w to about 90% w/w. In yet another aspect, the amount may be from about 50% w/w to about 80% w/w.

Another way of characterizing the amount of stabilizer used is in relation to the amount of hydrophobic drug present. When cyclosporin is the hydrophobic drug used, in one aspect, the ratio of stabilizer to hydrophobic drug may be at least about 5:1. In another aspect, the ratio may be at least about 6:1. In yet another aspect, the ratio may be at least about 7:1.

While the solubilizers and solubilizer compositions used in the present invention may include various sorbitan esters and other ingredients as noted above, in certain embodiments, such as when cyclosporine is used, some compounds may be specifically excluded for use as solubilizers. For example, in one aspect, the solubilizer and solubilizer compositions may be substantially free of lipophilic components, such as oils, triglycerides, or non-polyethoxylated surfactants having an HLB value of less than about 10. In another aspect, the solubilizer and solubilizer composition may be substantially free of sorbitan esters. In yet another aspect, such solubilizer and solubilizer compositions may be substantially free of polyethoxylated sorbitan fatty acid esters. In a further aspect, such solubilizer and solubilizer compositions may be substantially free of tocopherol or tocopherol derivatives, especially tocopherol polyethylene glycol succinate (TPGS).

In another aspect, the compositions and dispersions of the present invention may be substantially free of certain ingredients, regardless of the intended use thereof. In one aspect, the compositions and dispersions may be substantially free of lipophilic components, such as oils, triglycerides, or non-polyethoxylated surfactants having an HLB value of less than about 10. In another aspect, the compositions and dispersions may be substantially free of sorbitan esters. In yet another aspect, such the compositions and dispersions may be substantially free of polyethoxylated sorbitan fatty acid esters. In a further aspect, such the compositions and dispersions may be substantially free of tocopherol or tocopherol derivatives, especially tocopherol polyethylene glycol succinate (TPGS).

D. Composition and Dispersion Characteristics

The compositions of the present invention form an aqueous dispersion of the hydrophobic drug upon mixing with an aqueous medium (5-500× dilution w/w and particularly 10-250×w/w dilution), including without limitation gastric fluids and simulated gastric fluids, purified or deionized water, and buffers. As recited above, such dispersions contain the hydrophobic drug in both a dissolved form, as well as particles of undissolved drug. The particles of undissolved drug may comprise the active agent(s) only and may further comprises the stabilizer, the solubilizer and/or any additive in the particles and/or on the surface of the particles at various ratios. It has been found that such dispersions, containing both dissolved and undissolved drug are capable of providing bioavailability of the drug that is in many cases comparable to or better than dispersions in which the drug is completely dissolved. In fact, with regard to cyclosporine, a dosage form comprising the compositions of the present invention may attain a cyclosporine blood area under the curve (AUC) value of from about 60% to about 150% and a Cₘₐₓ of from about 60% to about 150% of those attained from a formulation containing cyclosporine as a microemulsion pre-concentrate. In an additional aspect, the AUC may be from about 80% to about 125%, and the Cₘₐₓ may be from about 80% to about 125%. Notably, such values may be obtained upon administration of the present composition to a subject, regardless of whether the administration occurs with food, or without food. One example of a currently commercialized product with cyclosporine formulated as a microemulsion preconcentrate is the currently marketed by Novartis Pharmaceuticals Corporation (East Hanover, N.J.) under the trade name Neoral® as recited above, the details of which may be found on pages 2380-2387 of The Physicians' Desk Reference, 56^{th} ed. (2002), which is incorporated herein by reference.

Without wishing to be bound by theory, it is generally thought that as dissolved drug becomes absorbed by the biological system, that room is created in the dispersion for the solubilization of the undissolved particles of drug. As the absorption of drug by the biological system is a continuous process, drug is continually moving from the undissolved particle state to a dissolved state, until such time as absorption stops, or the supply of undissolved drug particles is exhausted. It is also thought that the dissolved fraction (i.e. loading dose) will be absorbed rapidly to give faster onset and efficacy, and the undissolved fraction (i.e. maintanence dose) is made available in the form of continued relief/efficacy.

The proportion of hydrophobic drug that is contained in dissolved form, as compared to the undissolved particles of the dispersion may vary. When the hydrophobic drug is cyclosporine, in one aspect, at least about 35% w/w of the drug is dissolved. In another aspect, at least about 50% w/w of the cyclosporine is dissolved. Conversely, in one aspect, the amount of the cylcosporine that remains as undissolved particles may be at least about 20% w/w. In another aspect, the amount may be at least about 30% w/w. Moreover, it has been found that the amount of dissolved drug can be effectively controlled by the amount of stabilizer in the formulation as recited above. Accordingly, as the amount of stabilizer increases, so does the amount of drug which becomes dissolved in the dispersion.

The dissolved portion of the hydrophobic drug in the dispersion may exist to some extent as directly dissolved in the bulk aqueous phase. Still more dissolved drug may be contained in droplets or particles of solubilized drug, such as micelles which also remain associated with the bulk aqueous phase. This phenomenon in combination with the particles containing solid, or undissolved drug may have the effect of creating a bimodal particle phenomenon within the dispersion (i.e. bimodal distribution). That is to say, that multiple populations of particles and/or droplets may be identified based on differences in the mean diameters thereof, typically differences of at least about 10 nm to about 100 nm (the mean diameter is obtained based on volume weighed distribution). In one aspect, the difference in mean diameters may be at least about 10 nm. In another aspect, the difference may be at least about 20 nm. In yet another aspect, the difference may be at least about 50 nm. In a further aspect, the difference may be at least about 100 nm.

The mean diameters of the multiple population of particles or droplets may be measured through a number of techniques known to those of ordinary skill in the art, such as light scattering techniques, etc. In one aspect, of the invention, the droplets and the undissolved particles may represent at least two distinct populations of diameter size. In another aspect, additional size populations may be formed within each of the respective droplet and undissolved particle communities.

Various mechanisms may be employed to identify and characterize the existence of multiple size populations in the dispersions of the present invention. In one aspect, multiple populations may be identified by filtering the dispersion through a membrane having pores that are smaller than the diameter of the particles of undissolved drug contained in the dispersion. In this case, the undissolved particles of drug will be retained on the membrane or filter, and the dissolved drug droplets will pass through the membrane and remain with the bulk aqueous phase. In one aspect, the undissolved drug particles may have an average diameter such that the particles of undissolved cyclosporine are retained on membranes with mean pore diameter of about 0.2 um.

Another mechanism for determining the existence of multiple droplet and particle size populations is centrifugation. By centrifuging the dispersions of the present invention at a sufficient centrifugation force for a sufficient time, the undissolved drug particles of sufficient size will form a pellet or settle at the bottom of the centrifugation tube. By contrast, the solubilized or dissolved drug associated with droplets and smaller particles will remain buoyant with the bulk aqueous phase. A variety of specific centrifugation parameters may be used for such a quantification as will be recognized by those of ordinary skill in the art. However, in one aspect, the undissolved drug particles is identified from the dispersion by centrifuging the dispersion at about 12K×G for about 10 minutes. It is to be noted that in some circumstances, even if multiple size populations of droplets and undissolved particles are not identified, that testing in the above-recited manners may simply confirm the existence of both dissolved drug and undissolved drug particles in the dispersion.

Because of the undissolved particles of drug, and because of the droplet form in which the dissolved cyclosporine may be contained, the dispersion of the present invention is typically turbid. Of course the specific turbidity of the formulation will depend on the ratio of dissolved drug to undissolved drug. However, in the case of cyclosporine, in one aspect of the present invention, the dispersion from a 100×w/w dilution of the composition in an aqueous medium may have a turbidity that is sufficient to provide a UV absorption of at least about 0.5 at a wavelength of about 400 nm through a 1 cm thick cell at ambient temperature. In another aspect, the absorption may be at least about 1. In yet another aspect, the absorption may be greater than about 2 at 400 nm. Such values are clearly higher than for typical microemulsions in which cyclosporine is completely solubilized.

The particle size and distribution of the undissolved fraction can be customized for desirable bioperformance. The biomodal or multimodal distribution can be customized through judicious choice of stabilizer(s) for the respective drugs and intended bioavailability/efficacy.

The aqueous dispersions of the present invention are stable for an amount of time that is sufficient to allow absorption of the relevant hydrophobic drug. In the case of cyclosporine, the aqueous dispersion is stable (i.e. the undissolved cyclosporine particles are prevented from settlement by the stabilizer) for at least about 2-4 hours after dispersion of the pre-concentrate composition in the aqueous medium. Such stability of the undissolved cyclosporine can be characterized as at least 60%, more preferably 80% w/w of the cyclosporine remaining associated with the bulk aqueous medium when the dispersion is allowed to stand still for about 2-4 hours.

The compositions of the present invention may be provided in a variety of dosage forms for administration to a patient having a condition for which the specific hydrophobic drug is indicated. However, in one aspect, the dosage form may be an oral dosage form. Examples of suitable oral dosage forms include both hard and soft gelatin capsule forms, as well as tablet forms, liquids, and oral suspensions or syrups. In one aspect, the dosage form may be a gelatin capsule. In another aspect, the gelatin capsule may be a soft gelatin capsule. Of course, those of ordinary skill in the art will be able to determine the specific ingredients and drug loading required to formulate a given dosage form, while still remaining within the spirit and scope intended by the present invention.

E. Additives

In addition to the above-recited components, the compositions and dispersions of the present invention may include a variety of other ingredients as required in order to produce a specific dosage form, or attain a specifically desired result. Those of ordinary skill in the art will be able to determine such ingredients without undue experimentation. For example, in one aspect, the present formulations may include a solidifier or thickener in order to improve the compatibility of the formulation with certain oral dosage forms, such as capsule dosages. The presence of a solidifier or thickening agent has been found to reduce the incidence of leakage from capsules during encapsulation and storage. Moreover, such an ingredient may reduce evaporation of volatile solvents, such as ethanol during processing and storage, thus improving the stability thereof.

F. Methods

Those of ordinary skill in the art will fully appreciate the variety of methods by which the compositions recited herein may be made. Specific procedures will of course, depend on the type of dosage form being fabricated, and those of ordinary skill in the art will be able to readily adapt various known methods to include the specific ingredients and amounts therefor as set out herein. In one aspect, the procedure for fabrication of cyclosporine compositions may generally follow the steps of: dissolving the cyclosporine in the solubilizer, and adding the stabilizer to form a homogenous mixture. At this point, other additives may be introduced into the composition as required to obtain a specific dosage form. Additionally, processing conditions may give consideration to the types of ingredients being added to the composition. For example, in some cases, the preparation of a composition may proceed using reduced temperatures in order to minimize the loss of volatile solvents used as the solubilizer.

In addition, the present invention encompasses methods for the use thereof. Those of ordinary skill in the art will recognize specific physiological conditions for which a given hydrophobic drug may be indicated as treatment. Generally speaking, one method of the present invention may include the steps of providing a pharmaceutical composition as recited herein and administering such composition in a therapeutically effective amount to a subject having a condition for which the specific hydrophobic drug is indicated.

In one aspect, cyclosporine may be used to treat a condition in a subject for which cyclosporine is indicated. Such a method may include providing a cyclosporine composition as recited herein, and administering the composition to the subject in a therapeutically effective amount.

The following examples are presented for the purposes of illustrating various possible embodiments of the present invention, in order to provide those of ordinary skill in the art with an additional understanding of the inventive concepts involved therein. As such, it is understood that no limitation on the scope or content of the invention is to be perceived therefrom.

### EXAMPLES

Compositions in accordance with certain embodiments of the present invention were prepared by combining cyclosporine or other hydrophobic active agents with the specified excipients in specific proportions as enumerated below. For each composition, cyclosporine, the solubilizer and the lipidic stabilizer(s) can be combined at the same time or in any order to form a homogenous mixture in which cyclosporine is dissolved completely. However, in one aspect, cyclosporine can be combined with the solubilizer first to be dissolved completely and then the lipidic stabilizer can be added to form a homogenous mixture.

When a solid or semi-solid composition is desired, an additional solidifier/thickener can be introduced to the composition. The solidifier/thickener can be introduced at an elevated temperature to facilitate the formation of a homogenous mixture. However, if the composition includes a volatile solvent, such as ethanol, it is recommended that the solidifier/thickener be introduced last in order to minimize the loss of the solvent to evaporation during the process.

According to the above procedure, the compositions of Examples 1-60 were prepared. Notably, the proportion and amount of active agent may vary depending on therapeutic dose and desired unit dosage form and size of the dosage form. Moreover, it is to be understood that various active ingredients may be substituted for the cyclosporine active ingredient in the below examples. However, as will be recognized by one of ordinary skill in the art, different active agents that are substituted for the cyclosporine may be required in different amounts than those specifically recited for cyclosporine. Therefore, the numerical amounts in the examples have been recited in parts by weight in order to allow greater flexibility in substituting active agents.

The examples listed below are typical and not limiting with respect to active levels compared to non-active component levels. The dose range of cyclosporine present in an unit dosage form, such as a capsule, containing the compositions described in the following examples can be from 10-200 mg, or more preferably, from 25-100 mg. As will be seen, the cyclosporine in the examples can also be replaced by another active agent, such as tacromilus, sirolimus, dutesteride, cilostazol, progesterone, fenofibrate, amiodarone, spironolactone, budesonide, carosprodol, celecoxib, atorvastatin, glimepiride, saquinavir, ritanovir, everolimus, nefazodone, metaxalone, pimecrolimus, tazarotene, valproic acid, nilutamide, or bicalutamide.

### Example 1

| Component | w/w (in parts) |
|---|---|
| Cyclosporine | 12.0 |
| Ethanol | 8.8 |
| Cremophor EL | 39.6 |
| Cremophor RH40 | 39.6 |

### Example 2

| Component | w/w (in parts) |
|---|---|
| Cyclosporin | 12.0 |
| Ethanol | 11.0 |
| Cremophor EL | 38.5 |
| Cremophor RH40 | 38.5 |

### Example 3

| Component | w/w (in parts) |
|---|---|
| Cyclosporin | 12.0 |
| Ethanol | 13.2 |
| Cremophor EL | 37.4 |
| Cremophor RH40 | 37.4 |

### Example 4

| Component | w/w (in parts) |
|---|---|
| Cyclosporin | 12.0 |
| Ethanol | 15.4 |
| Cremophor EL | 36.3 |
| Cremophor RH40 | 36.3 |

### Example 5

| Component | w/w (in parts) |
|---|---|
| Cyclosporin | 11.0 |
| Ethanol | 15.4 |
| Cremophor EL | 36.8 |
| Cremophor RH40 | 36.8 |

### Example 6

| Component | w/w (in parts) |
|---|---|
| Cyclosporin | 10.0 |
| Ethanol | 15.4 |
| Cremophor EL | 37.3 |
| Cremophor RH40 | 37.3 |

### Example 7

| Component | w/w (in parts) |
|---|---|
| Cyclosporin | 9.0 |
| Ethanol | 15.4 |
| Cremophor EL | 37.8 |
| Cremophor RH40 | 37.8 |

### Example 8

| Component | w/w (in parts) |
|---|---|
| Cyclosporin | 10.0 |
| Ethanol | 7.7 |
| Cremophor EL | 37.3 |
| Cremophor RH40 | 37.3 |

### Example 9

| Component | w/w (in parts) |
|---|---|
| Cyclosporin | 10.0 |
| Ethanol | 15.4 |
| Cremophor EL | 74.6 |

### Example 10

| Component | w/w (in parts) |
|---|---|
| Cyclosporin | 10.0 |
| Ethanol | 15.4 |
| Cremophor EL | 56.0 |
| Cremophor RH40 | 18.6 |

### Example 11

| Component | w/w (in parts) |
|---|---|
| Cyclosporin | 10.0 |
| Ethanol | 15.4 |
| Cremophor EL | 18.6 |
| Cremophor RH40 | 56.0 |

### Example 12

| Component | w/w (in parts) |
|---|---|
| Cyclosporin | 10.0 |
| Ethanol | 15.4 |
| Cremophor RH40 | 74.6 |

### Example 13

| Component | w/w (in parts) |
|---|---|
| Cyclosporin | 10.0 |
| Ethanol | 15.4 |
| Cremophor EL | 37.3 |
| Cremophor RH40 | 37.3 |
| Water | 5.0 |

### Example 14

| Component | w/w (in parts) |
|---|---|
| Cyclosporin | 10.0 |
| Ethanol | 15.4 |
| Cremophor EL | 37.3 |
| Cremophor RH40 | 37.3 |
| Water | 10.0 |

### Example 15

| Component | w/w (in parts) |
|---|---|
| Cyclosporin | 10.0 |
| Ethanol | 15.4 |
| Cremophor EL | 37.3 |
| Cremophor RH40 | 37.3 |
| Water | 15.0 |

### Example 16

| Component | w/w (in parts) |
|---|---|
| Cyclosporin | 10.0 |
| Ethanol | 10.8 |
| Cremophor EL | 37.3 |
| Cremophor RH40 | 37.3 |
| Water | 10.0 |

### Example 17

| Component | w/w (in parts) |
|---|---|
| Cyclosporin | 10.0 |
| Ethanol | 10.8 |
| Cremophor EL | 37.3 |
| Cremophor RH40 | 37.3 |
| Water | 15.0 |

### Example 18

| Component | w/w (in parts) |
|---|---|
| Cyclosporin | 10.0 |
| Ethanol | 7.7 |
| Cremophor EL | 37.3 |
| Cremophor RH40 | 37.3 |
| Water | 5.0 |

### Example 19

| Component | w/w (in parts) |
|---|---|
| Cyclosporin | 10.0 |
| Ethanol | 13.5 |
| Labrasol | 4.5 |
| Cremophor EL | 36.0 |
| Cremophor RH40 | 36.0 |

### Example 20

| Component | w/w (in parts) |
|---|---|
| Cyclosporin | 10.0 |
| Ethanol | 13.5 |
| Labrasol | 6.0 |
| Cremophor EL | 35.25 |
| Cremophor RH40 | 35.25 |

### Example 21

| Component | w/w (in parts) |
|---|---|
| Cyclosporin | 10.0 |
| Ethanol | 13.5 |
| Labrasol | 9.0 |
| Cremophor EL | 33.75 |
| Cremophor RH40 | 33.75 |

### Example 22

| Component | w/w (in parts) |
|---|---|
| Cyclosporin | 10.0 |
| Ethanol | 13.5 |
| Labrasol | 18.0 |
| Cremophor EL | 29.25 |
| Cremophor RH40 | 29.25 |

### Example 23

| Component | w/w (in parts) |
|---|---|
| Cyclosporin | 10.0 |
| Ethanol | 13.5 |
| Labrasol | 50.0 |
| Cremophor EL | 13.25 |
| Cremophor RH40 | 13.25 |

### Example 24

| Component | w/w (in parts) |
|---|---|
| Cyclosporin | 10.0 |
| Ethanol | 13.5 |
| Gelucire 44/14 | 76.5 |

### Example 25

| Component | w/w (in parts) |
|---|---|
| Cyclosporin | 10.0 |
| Ethanol | 13.5 |
| Gelucire 44/14 | 54.0 |
| Cremophor EL | 11.25 |
| Cremophor RH40 | 11.25 |

### Example 26

| Component | w/w (in parts) |
|---|---|
| Cyclosporin | 10.0 |
| Ethanol | 13.5 |
| Gelucire 44/14 | 36.0 |
| Cremophor EL | 20.25 |
| Cremophor RH40 | 20.25 |

### Example 27

| Component | w/w (in parts) |
|---|---|
| Cyclosporin | 10.0 |
| Ethanol | 13.5 |
| Gelucire 44/14 | 18.0 |
| Cremophor EL | 29.25 |
| Cremophor RH40 | 29.25 |

### Example 28

| Component | w/w (in parts) |
|---|---|
| Cyclosporin | 12.0 |
| Ethanol | 13.2 |
| Cremophor EL | 44.0 |
| Labrafil M2125CS | 30.8 |

### Example 29

| Component | w/w (in parts) |
|---|---|
| Cyclosporin | 12.0 |
| Ethanol | 13.2 |
| Cremophor EL | 44.0 |
| Labrafil M2125CS | 30.8 |
| PEG 8000 | 1.5 |

### Example 30

| Component | w/w (in parts) |
|---|---|
| Cyclosporin | 12.0 |
| Ethanol | 13.2 |
| Cremophor EL | 44.0 |
| Labrafil M2125CS | 30.8 |
| PEG 8000 | 5.0 |

### Example 31

| Component | w/w (in parts) |
|---|---|
| Cyclosporin | 13.0 |
| Ethanol | 15.4 |
| Cremophor EL | 43.0 |
| Labrafil M2125CS | 28.6 |

### Example 32

| Component | w/w (in parts) |
|---|---|
| Cyclosporin | 13.0 |
| Ethanol | 15.4 |
| Cremophor EL | 43.0 |
| Labrafil M2125CS | 28.6 |
| PEG 8000 | 5.0 |

### Example 33

| Component | w/w (in parts) |
|---|---|
| Cyclosporin | 13.0 |
| Ethanol | 15.4 |
| Propylene Glycol | 4.6 |
| Cremophor EL | 40.0 |
| Labrafil M2125CS | 27.0 |

### Example 34

| Component | w/w (in parts) |
|---|---|
| Cyclosporin | 10.0 |
| Propylene Glycol | 15.4 |
| Cremophor EL | 37.3 |
| Cremophor RH40 | 37.3 |

### Example 35

| Component | w/w (in parts) |
|---|---|
| Cyclosporin | 11.0 |
| Ethanol | 10.0 |
| Propylene Glycol | 10.0 |
| Cremophor EL | 34.5 |
| Cremophor RH40 | 34.5 |

### Example 36

| Component | w/w (in parts) |
|---|---|
| Cyclosporin | 10.0 |
| Ethanol | 15.4 |

### Example 37

| Component | w/w (in parts) |
|---|---|
| Bicalutamide | 10 |
| Ethanol | 8.8 |
| Cremophor EL | 40.6 |
| Cremophor RH40 | 40.6 |

### Example 38

| Component | w/w (in parts) |
|---|---|
| Nilutamide | 16.0 |
| Ethanol | 11.0 |
| Cremophor EL | 36.5 |
| Cremophor RH40 | 36.5 |

### Example 39

| Component | w/w (in parts) |
|---|---|
| Tacrolimus | 2.0 |
| Ethanol | 13.2 |
| Cremophor EL | 42.4 |
| Cremophor RH40 | 42.4 |

### Example 40

| Component | w/w (in parts) |
|---|---|
| Sirolimus | 2.0 |
| Ethanol | 15.4 |
| Cremophor EL | 41.3 |
| Cremophor RH40 | 41.3 |

### Example 41

| Component | w/w (in parts) |
|---|---|
| Bicalutamide | 10 |
| Ethanol | 15.4 |
| labrasol | 74.6 |

### Example 42

| Component | w/w (in parts) |
|---|---|
| Fenofibrate | 10.0 |
| Ethanol | 15.4 |
| Cremophor EL | 37.3 |
| Cremophor RH40 | 37.3 |

### Example 43

| Component | w/w (in parts) |
|---|---|
| Progesterone | 9.0 |
| Ethanol | 15.4 |
| Cremophor EL | 37.8 |
| Cremophor RH40 | 37.8 |

### Example 44

| Component | w/w (in parts) |
|---|---|
| Cilostazol | 10.0 |
| Ethanol | 7.7 |
| Cremophor EL | 37.3 |
| Cremophor RH40 | 37.3 |

### Example 45

| Component | w/w (in parts) |
|---|---|
| Amiodarone | 10.0 |
| Ethanol | 15.4 |
| Cremophor EL | 74.6 |

### Example 46

| Component | w/w (in parts) |
|---|---|
| Dutasteride | 1.0 |
| Ethanol | 15.4 |
| Cremophor EL | 56.0 |
| Cremophor RH40 | 28.6 |

### Example 47

| Component | w/w (in parts) |
|---|---|
| Spironolactone | 10.0 |
| Ethanol | 15.4 |
| Cremophor EL | 18.6 |
| Cremophor RH40 | 56.0 |

### Example 48

| Component | w/w (in parts) |
|---|---|
| Dutasteride | 1 |
| Ethanol | 15.4 |
| Cremophor RH40 | 84.6 |

### Example 49

| Component | w/w (in parts) |
|---|---|
| Metaxalone | 10.0 |
| Ethanol | 15.4 |
| Cremophor EL | 37.3 |
| Cremophor RH40 | 37.3 |
| Water | 5.0 |

### Example 50

| Component | w/w (in parts) |
|---|---|
| Celecoxib | 10.0 |
| Ethanol | 15.4 |
| Cremophor EL | 37.3 |
| Cremophor RH40 | 37.3 |
| Water | 10.0 |

### Example 51

| Component | w/w (in parts) |
|---|---|
| Atorvastatin | 10.0 |
| Ethanol | 15.4 |
| Cremophor EL | 37.3 |
| Cremophor RH40 | 37.3 |
| Water | 15.0 |

### Example 52

| Component | w/w (in parts) |
|---|---|
| Atorvastatin | 10.0 |
| Ethanol | 10.8 |
| Cremophor EL | 42.3 |
| Cremophor RH40 | 42.3 |

### Example 53

| Component | w/w (in parts) |
|---|---|
| Saquinavir | 15.0 |
| Ethanol | 10.8 |
| Cremophor EL | 42.3 |
| Cremophor RH40 | 42.3 |

### Example 54

| Component | w/w (in parts) |
|---|---|
| Valproic acid | 10.0 |
| Ethanol | 7.7 |
| Cremophor EL | 37.3 |
| Cremophor RH40 | 37.3 |
| Water | 5.0 |

### Example 55

| Component | w/w (in parts) |
|---|---|
| Nefazodone | 10.0 |
| Ethanol | 13.5 |
| Labrasol | 4.5 |
| Cremophor EL | 36.0 |
| Cremophor RH40 | 36.0 |

### Example 56

| Component | w/w (in parts) |
|---|---|
| Tazarotene | 10.0 |
| Ethanol | 13.5 |
| Labrasol | 6.0 |
| Cremophor EL | 35.25 |
| Cremophor RH40 | 35.25 |

### Example 57

| Component | w/w (in parts) |
|---|---|
| Ritanovir | 10.0 |
| Ethanol | 13.5 |
| Labrasol | 9.0 |
| Cremophor EL | 33.75 |
| Cremophor RH40 | 33.75 |

### Example 58

| Component | w/w (in parts) |
|---|---|
| Carosprodol | 10.0 |
| Ethanol | 13.5 |
| Labrasol | 18.0 |
| Cremophor EL | 29.25 |
| Cremophor RH40 | 29.25 |

### Example 59

| Component | w/w (in parts) |
|---|---|
| Dronabinol | 10.0 |
| Ethanol | 13.5 |
| Labrasol | 50.0 |
| Cremophor EL | 13.25 |
| Cremophor RH40 | 13.25 |

### Example 60

The dissolution of cyclosporine from the compositions of the present invention was evaluated. The release of cyclosporine from each composition of Examples 3-7, 36 and control sample (Neoral® oral capsule) in simulated gastric fluid (SGF) at 37° C. were compared. The dissolution was conducted in a USP type I (rotating basket at 100 rpm) dissolution apparatus with 250 ml of SGF. At t=30 min. and 2 hr., an aliquot of the dissolution medium was removed and subjected to particle size analysis. The volume-weighted bimodal particle size distribution was analyzed by dynamic light scattering (NICOMP particle sizer). Another aliquot was also withdrawn and filtered through a 0.2 μm membrane filter or centrifuged at ˜12 K×G for 10 min. at ambient temperature. The resulting clear filtrate and supernatant were subject to HPLC assay for determining the cyclosporine concentration to determine the fraction of cyclosporine that remained solubilized in the dissolution medium. The fraction of cyclosporine precipitated in the dissolution medium was subsequently calculated. Alternatively, the fraction of cyclosporine precipitated in the dissolution medium was determined by the cyclosporine content in the pellet obtained from the centrifuged samples. The results are summarized in the following tables:

TABLE 19
Particle Size Distribution of Aqueous Dispersion from
Cyclosporine-Containing Pre-Concentrate

| | Particle Size Distribution at t = 30 min; | |
|---|---|---|
| Example 3 | Unfiltered Sample | Filtrate (0.2 μm) |
| Mean diameter ± SD | 122.2 ± 16.4 (24.7%) | 11.8 ± 2.4 (99.9%) |
| (Volume distribution) | 359.9 ± 677 (75.3%) | 312.5 ± 3.0 (0.1%) |
| % of Cyclosporin | (100%) | 50.9% |
| Remains Association | | |
| with the Fraction | | |

TABLE 20
Percentage of Solubilized Cyclosporine in Aqueous Dispersions
from the Compositions of the Present Invention and Prior Art

| | |
|---|---|
| | % of Cyclosporin Solubilized |
| Example | in the Dissolution Medium |

| Number | t = 30 min. | t = 2 hr. |
|---|---|---|
| 4 | 39.6 | — |
| 5 | 50.3 | 53.6 |
| 6 | 54.5 | 55.2 |
| 7 | 64.1 | 64.3 |
| 36 | 0.2 | 0.2 |
| Neoral ® | 97.6 | — |

At a ratio of the stabilizer to cyclosporine of about 6:1 (6.23:1 for Example 3), no settling of the suspended solid particle containing cyclosporine was observed over at least 2 hours. Even though the mean diameter of these solid particles comprising cyclosporine from the composition of the present invention is generally at least about 200 nm which is much greater than that of the microemulsion formed from Neoral® which is only about 30 nm in diameter and monodistributed, a relatively stable dispersion of cyclosporine was obtained as evidenced by the particle sizing data over the 2 hour period.

It is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A pharmaceutical composition comprising:
a testosterone ester in both dissolved form and as undissolved particles, said dissolved form comprising at least 35 wt % of the testosterone ester present in the composition;
a solubilizer; and
a stabilizer.

2. The pharmaceutical composition of claim 1, wherein the solubilizer is selected from the group consisting of ethanol, benzyl alcohol, propylene glycol, glycerol, polyglycerol, transcutol, polyethylene glycol, polypropylene glycol, polyvinylalcohol, hydroxypropyl methylcellulose, glycofurol, 2-pyrrolidone, polyvinylpyrrolidone, tributylcitrate, acetyl triethylcitrate, triethylcitrate, ethyl oleate, triacetin, propylene glycol monoacetate, propylene glycol diacetate, dimethyl isosorbide, and mixtures thereof.

3. The pharmaceutical composition of claim 1, wherein the solubilizer is selected from the group consisting of ethanol, benzyl alcohol, propylene glycol, glycerol, polyglycerol, polyethylene glycol, polyvinylalcohol, polyvinylpyrrolidone, tributylcitrate, triethylcitrate, ethyl oleate, triacetin, and mixtures thereof.

4. The pharmaceutical composition of claim 1, wherein the solubilizer comprises about 5 wt % to about 20 wt % of the composition.

5. The pharmaceutical composition of claim 1, wherein the solubilizer comprises about 7 wt % to about 16 wt % of the composition.

6. The pharmaceutical composition of claim 1, wherein the solubilizer is present in an amount of 10 wt % to 50 wt % with respect to the testosterone ester.

7. The pharmaceutical composition of claim 1, wherein the stabilizer is selected from the group consisting of polyethoxylated fatty acids, PEG-fatty acid diesters, polyethylene glycol glycerol fatty acid esters, alcohol-oil transesterification products, polyglycerized fatty acids, mono- and diglycerides, glycerol esters of fatty acids, and mixtures thereof.

8. The pharmaceutical composition of claim 1, wherein the stabilizer is selected from the group consisting of polyethyoxylated castor oils, glycerol monolinoleate, glyceryl monostearate, glyceryl caprylate-caprate, glyceryl palmitic/stearic acid, and mixtures thereof.

9. The pharmaceutical composition of claim 1, wherein the stabilizer includes mixtures of polyoxyl 35 castor oil and polyoxyl 40 hydrogenated castor oil.

10. The pharmaceutical composition of claim 1, wherein the stabilizer comprises about 10 wt % to about 90 wt %.

11. The pharmaceutical composition of claim 1, wherein the testosterone ester comprises about 1 wt % to about 20 wt % of the composition.

12. The pharmaceutical composition of claim 1, wherein the composition forms an aqueous dispersion upon mixing with an aqueous medium.

13. The pharmaceutical composition of claim 12, wherein the testosterone ester is present in dissolved form and as undissolved particles in the aqueous dispersion.

14. The pharmaceutical composition of claim 12, wherein at least 20 wt % of the testosterone ester is present as undissolved particles in the aqueous dispersion.

15. The pharmaceutical composition of claim 13, wherein the aqueous dispersion of the composition provides substantially equivalent bioavailability as compared to a dispersion having only dissolved drug.

16. The pharmaceutical composition of claim 13, wherein the difference in the mean diameter measured based on volume weighed distribution of the particle and/or droplet containing the dissolved testosterone ester and the undissolved testosterone ester in the aqueous dispersion is at least about 10 nm.

17. The pharmaceutical composition of claim 1, wherein the stabilizer is substantially free of lipophilic components.

18. The pharmaceutical composition of claim 1, wherein the stabilizer is substantially free of polyoxylethylene sorbitan fatty acid ester or sorbitan fatty acid ester.

19. The pharmaceutical composition of claim 1, wherein the stabilizer is substantially free of TPGS.

20. The pharmaceutical composition of claim 1, wherein the composition further comprises a thickening agent.

21. The pharmaceutical composition of claim 1, wherein the composition is an oral dosage form.

22. The pharmaceutical composition of claim 1, wherein the oral dosage form is a soft gelatin capsule.
